(19) United States
(12) Patent Application Publication
(10) Pub. No.: US 2025/0006293 A1
(43) Pub. Date: Jan. 2, 2025

(54) METHODS FOR ANTIBODY OPTIMIZATION

(71) Applicant: Vibrant Pharma Limited, Guangzhou (CN)

(72) Inventors: Bojian Xiong, Guangzhou (CN); Chunfa Dong, Guangzhou (CN); Hong Zhang, Guangzhou (CN); Luquan Wang, Guangzhou (CN)

(21) Appl. No.: 18/698,817

(22) PCT Filed: Nov. 4, 2022
(86) PCT No.: PCT/CN2022/130024

§ 371 Date: Apr. 5, 2024

(30) Foreign Application Priority Data
Nov. 4, 2021 (WO) PCT/CN2021/128573

(51) Int. Cl. G16B 15/20; G06F 30/27; G16B 20/50; G16B 40/00

(52) U.S. Cl. CPC G16B 15/20; G06F 30/27; G16B 20/50; G16B 40/00

(57) ABSTRACT

Provided are methods for antibody engineering. In one aspect, the methods involve a computer-aided method for high-efficiency antibody engineering optimization.

### TECHNICAL FIELD

The disclosure relates to methods of antibody engineering.

### BACKGROUND

An antibody (also known as an immunoglobulin) is a protein produced by the immune system. It can recognize foreign objects such as pathogenic bacteria and viruses. Antigen-specific antibodies can be produced by injecting an antigen into a mammal, such as a mouse, rat, rabbit, goat, sheep, camel, alpaca, lamma, or horse. Blood isolated from these animals contains polyclonal antibodies in the serum. To obtain monoclonal antibodies, antibody-secreting lymphocytes are isolated from the animal and immortalized by fusing them with a cancer cell line. The fused cells are called hybridomas, and will continually grow and secrete monoclonal antibodies in culture. Monoclonal antibodies can also be obtained using techniques like B cell cloning and phage display library screening.

Antibodies can be used to treat various diseases in human, e.g., cancer, autoimmune diseases, Alzheimer's disease, etc. However, antibodies obtained from animals (such as mice, rabbits, apes or camels), if administered directly to human patients, usually lead to the production of human neutralizing antibodies against these antibodies, making the antibodies obtained or derived from animals less effective. Humanizing these antibodies is often required to address this problem. Traditional methods for humanizing antibodies require time-consuming and labor-intensive experiments to obtain engineered antibodies with improved immunogenicity and antigen binding affinity. More efficient methods for humanizing and engineering antibodies are needed.

### SUMMARY

The present disclosure provides computer-aided methods for high-efficiency antibody engineering optimization. Traditional antibody engineering (such as humanization, optimization of engineering properties, optimization of manufacturability, etc.) requires multiple rounds of experiments for optimization. The present disclosure utilizes precise antibody structure prediction and structural difference comparison for high-efficiency antibody engineering optimization.

In one aspect, the disclosure is related to a computer-implemented method for producing an engineered antibody, the method comprising:
(a) generating, in silico, an engineered antibody by introducing one or more mutations to a starting antibody sequence;
(b) determining a spatial structure difference (SSD) in CDR between the engineered antibody and the starting antibody;
(c) determining the engineered antibody having a spatial structure difference that meets a threshold level; and
(d) selecting the engineered antibody.

In some embodiments, the spatial structure difference is predicted by a template free protein folding method.

In some embodiments, the spatial structure difference is predicted by a deep learning based protein folding method.

In some embodiments, the method comprises querying an input amino acid sequence of the engineered antibody in a database by neural network; and constructing a protein structure directly from the neural network.

In some embodiments, the SSD is measured by Root Mean Squared Deviation (RMSD), Template Modeling (TM) score, or IDDT score.

In some embodiments, the SSD is measured by RMSD.

In some embodiments, the RMSD is within 1 Å.

In some embodiments, the RMSD is within 2 Å.

In some embodiments, the RMSD is within 3 Å.

In some embodiments, the RMSD is within 4.5 Å.

In some embodiments, the spatial structure difference is selected from heavy chain CDR1SSD, the heavy chain CDR2 SSD, the heavy chain CDR3 SSD, the light chain CDR 1 SSD, the light chain CDR2 SSD, and the light chain CDR3 SSD.

In some embodiments, the spatial structure difference is calculated by combining two or more SSD values selected from: the heavy chain CDR1 SSD, the heavy chain CDR2 SSD, the heavy chain CDR3 SSD, the light chain CDR1 SSD, the light chain CDR2 SSD, and the light chain CDR3 SSD.

In some embodiments, the spatial structure difference is the sum of the heavy chain CDR1 SSD, the heavy chain CDR2 SSD, the heavy chain CDR3 SSD, the light chain CDR 1 SSD, the light chain CDR2 SSD, and the light chain CDR3 SSD.

In some embodiments, the spatial structure difference is the sum of the heavy chain CDR1 SSD, the heavy chain CDR2 SSD, and the heavy chain CDR3 SSD.

In some embodiments, the mutations are (a) point mutations or (b) replacing a part of the sequence of the starting antibody.

In some embodiments, framework sequences of the starting antibody are replaced by framework sequences of a human antibody.

In some embodiments, the engineered antibody is a humanized antibody.

In some embodiments, the engineered antibody has one or more improved antibody characteristics as comparing to the starting antibody.

In some embodiments, the engineered antibody comprises a sequence of an antibody that is selected from: AR1-1, AR1-2, AR1-3, AR1-4, AR1-5, AR1-6, AR1-7, AR1-9, AR1-10, AR1-11, BH-1, BH-2, BH-3, BH-4, BH-5, BH-6, BH-7, BH-8, BH-9, BH-10, BH-11, BH-12, BH-13, BH-14, BH-15, BH-16, BH-17, BH-18, BH-19, BH-20, BH-21, BH-22, BH-23, and BH-24.

In some embodiments, the method further comprises producing the engineered antibody.

In one aspect, the disclosure is related to a method of producing a humanized antibody, the method comprising:
(a) identifying CDR sequences and framework sequences of a non-human starting antibody;
(b) generating a plurality of humanized antibodies by replacing, in silico, the framework sequences of the starting antibody with the framework sequences of human germline antibodies;
(c) determining the spatial structure differences (SSD) between the plurality of humanized antibodies and the starting antibody; and
(d) selecting, a humanized antibody from the plurality of humanized antibodies with a spatial structure difference that meets a threshold value.

In some embodiments, the method comprises querying an input amino acid sequence of the plurality of humanized antibodies in a database by neural network; and constructing a protein structure directly from the neural network.

In some embodiments, the SSD is measured by RMSD.

In some embodiments, the RMSD is within 1 Å.

In some embodiments, the RMSD is within 2 Å.

In some embodiments, the RMSD is within 3 Å.

In some embodiments, the RMSD is within 4.5 Å.

In some embodiments, the spatial structure differences are calculated for each atom in the CDR sequences.

In some embodiments, the spatial structure differences are calculated for each non-H atom in the CDR sequences.

In some embodiments, the spatial structure differences are calculated for each atom in peptide bonds in the CDR sequences.

In some embodiments, the selected humanized antibody comprises a VHH.

In some embodiments, the spatial structure differences are calculated by combining VHH CDR1 SSD, VHH CDR2 SSD, and VHH CDR3 SSD.

In some embodiments, the spatial structure differences are calculated by adding one or more of VHH CDR1 SSD, VHH CDR2 SSD, and VHH CDR3 SSD.

In some embodiments, the selected humanized antibody comprises a VH and a VL.

In some embodiments, the spatial structure differences are calculated by combining the heavy chain CDR1 SSD, the heavy chain CDR2 SSD, the heavy chain CDR3 SSD, the light chain CDR 1 SSD, the light chain CDR2 SSD, and the light chain CDR3 SSD.

In some embodiments, the method further comprises introducing one or more mutations to the plurality of humanized antibodies.

In some embodiments, the binding affinity of the selected humanized antibody (KD) is less than 1×10⁻⁸ M.

In some embodiments, at least 100 or 1000 humanized antibodies are generated.

In some embodiments, the SSD between one humanized antibody and the starting antibody is determined within 1 minute.

In some embodiments, the method is completed within 1 day.

In some embodiments, the method further comprises making a vector comprising a nucleic acid sequence encoding the selected humanized antibody; and expressing the selected humanized antibody.

In some embodiments, the selected humanized antibody comprises a human constant region.

In one aspect, the disclosure is related to one or more machine-readable hardware storage devices for storing instructions that are executable by one or more data processing devices to perform the method described herein.

In one aspect, the disclosure is related to a system comprising: one or more data processing devices; and one or more machine-readable hardware storage devices that store instructions that are executable by the one or more data processing devices to execute the method described herein.

In some embodiments, the system further comprises one or more devices for making and expressing nucleic acid sequences.

In one aspect, the disclosure is related to a method for producing an engineered antibody, the method comprising:
(a) providing an amino acid sequence of an engineered antibody, wherein the engineered antibody is generated by the method comprising the following steps:
(i) generating, in silico, an engineered antibody by introducing one or more mutations to a starting antibody sequence;
(ii) determining a spatial structure difference (SSD) in CDR between the engineered antibody and the starting antibody, wherein the spatial structure is predicted by a template free protein folding method and/or deep learning based protein folding method;
(iii) determining the engineered antibody having a spatial structure difference that meets a threshold level; and
(b) producing the engineered antibody.

In some embodiments, the spatial structure difference is predicted by a template free protein folding method.

In some embodiments, the spatial structure difference is predicted by a deep learning based protein folding method.

In some embodiments, the method comprises querying an input amino acid sequence of the engineered antibody in a database by neural network; and constructing a protein structure directly from the neural network.

In some embodiments, the SSD is measured by RMSD, TM score, or IDDT score.

In some embodiments, the mutations are a) point mutations or b) replacing a part of the sequence of the starting antibody.

In some embodiments, framework sequences of the starting antibody are replaced by framework sequences of a human antibody.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain or VHH). Non-limiting examples of antibody fragments include, e.g., Fab, Fab′, F(ab′)2, and Fv fragments.

As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions (FR) are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “multimeric antibody” refers to an antibody that contains four or more (e.g., six, eight, or ten) immunoglobulin variable domains.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., PD-1) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a PD-1 molecule may be referred to as a PD-1-specific antibody or an anti-PD-1 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. The details of one or more embodiments of the invention are set forth in the accompanying drawings and the description below. Other features, objects, and advantages of the invention will be apparent from the description and drawings, and from the claims. The contents of any patents, patent applications, and other references cited in the specification are hereby incorporated by reference in their entirety.

### DESCRIPTION OF THE DRAWINGS

FIGS. 1A-1B illustrate the separation and purification of engineered antibodies expressed by host cells.

FIG. 2 illustrates a flowchart for an antibody engineering method in accordance with an embodiment of the present disclosure.

FIG. 3 shows the sequence, humanness score, spatial structure difference (root-mean-square deviation (RMSD), unit: Angstrom) and binding affinity (KD, unit: M) of the starting antibody (AR1-0) and engineered antibodies (AR1-1 to AR1-11).

FIGS. 4A-4K show the binding sensorgrams of the starting antibody (AR1-0) and engineered antibodies (AR1-1 to AR1-11).

FIG. 5 shows the sequence, humanness score, structural difference (RMSD, unit: Angstrom) and binding affinity (KD, unit: M) of the starting antibody (BH-0) and engineered antibodies (BH-1 to BH-24).

FIG. 6 is a schematic diagram showing a system for predicting protein structure, calculating the spatial structure difference (e.g., RMSD), calculating the humanness score, and selecting candidate engineered antibodies.

### DETAILED DESCRIPTION

Many processes in antibody engineering (such as humanization, optimization of expression, aggregation, manufacturability, viscosity, deamination sites, glycosylation sites, etc.) all require mutations in the antibody sequence. Traditional experimental methods require a large amount of time-consuming and labor-intensive screening experiments to obtain engineered antibodies that have both good affinity for the antigen and the desired engineering characteristics (e.g., expression, aggregation, manufacturability, viscosity, deamination sites, glycosylation sites, etc.).

In order to solve the shortcomings of high cost and reduce number of cycles in antibody screening in traditional experimental methods, the present disclosure provides a computer-aided antibody optimization method, which uses computers to predict the structural information of engineered antibodies (e.g., humanized antibodies) and to calculate its structural similarity with the original animal antibodies.

The traditional methods for humanization of non-human antibodies is complementary determining regions (CDR) grafting in which the CDRs of non-human antibodies are grafted onto the human frameworks. Typically, human frameworks with the highest homology to the framework regions of non-human antibody are chosen as an acceptor for CDR grafting. The problem associated with antibody humanization by this method is the loss of affinity to their specific targets. In many cases, CDR grafting results significant loss of binding affinity. The methods of CDR grafting is described in detail, e.g., Safdari, et al. “Antibody humanization methods-a review and update.” Biotechnology and Genetic Engineering Reviews 29.2 (2013): 175-186, which is incorporated herein by reference in its entirety. The traditional CDR grating methods focus on the homology of the framework sequence. While the human framework sequence may have a high homology with the framework sequence with the non-human antibodies, the difference in the framework sequence can still have a significant impact on the conformation of the CDR loop. Thus, CDR grafting often requires further time-consuming and labor-intensive experiments, e.g., affinity maturation.

The present disclosure involves protein structure prediction methods based on e.g., deep learning, and/or focuses on the CDR loop structure instead. Traditional protein structure prediction methods, such as homology modeling, use the skeleton structure of homologous proteins to predict the three-dimensional structure of the input amino acid primary sequence. These methods have a poor predictive performance on antibody structure, especially for the CDR loops. Deep learning-based folding algorithms, e.g., VibrantFold, AlphaFold2, and RoseTTaFold, can be used to predict the structure of a protein, particularly the structure of CDR loops. The method as described herein can greatly accelerate the speed of optimizing engineered antibodies, identify engineered antibodies that have the required binding affinity, and reduce the cost for drug development.

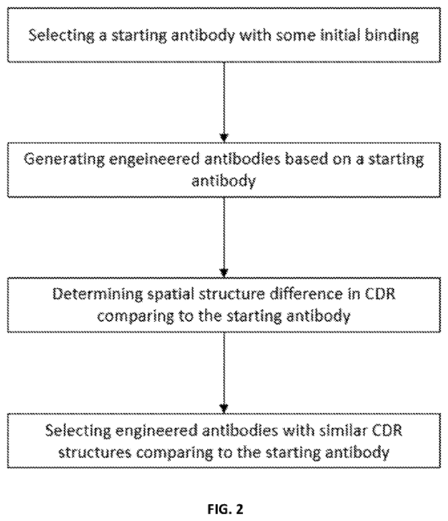

In one aspect, the general antibody engineering scheme is shown in FIG. 2. The steps in FIG. 2 are described in more detail below.

First, a starting antibody or a plurality of starting antibodies (e.g., non-human antibodies), with the desired property (e.g., function, or binding affinity) are used as a starting point.

Second, engineered antibodies are generated by making one or more amino acid changes (e.g., insertions, deletions, or substitutions) to the sequence of the starting antibody. These changes can be within either the CDR and/or the framework region of heavy chain and/or the light chain of the antibody.

Third, the spatial structure difference between the CDR of the starting antibody and each of the engineered antibodies is determined.

Then, the engineered antibodies with similar CDR structures comparing to the starting antibody (e.g., low spatial structure difference between the CDR of the starting antibody and the engineered antibody) are selected as promising candidates.

In some embodiments, in the case in which the antibody is being humanized. The disclosed method can include one or more of the following steps:
1) Obtaining the sequence of the animal-derived starting antibody through methods such as immunization as described herein;
2) Replacing the framework sequence of the above-mentioned starting sequence with the framework sequence of a variety of human antibody sequences (germline framework). At the same time, virtual point mutations can also be introduced into the engineered antibody sequences to obtain a series of engineered sequence modifications;
3) Using the structure prediction software to calculate the three-dimensional structure of the engineered antibodies using the engineered sequences obtained in step 2);
4) Comparing the three-dimensional structure of the variable region of the engineered antibodies obtained in step 3) with the three-dimensional structure of the variable region of the original animal antibody (the starting antibody), and calculating the spatial difference, and the optionally sorting the spatial differences between the variable region of the engineered antibodies and variable region the original animal antibody, and selecting one or several antibodies with the smaller spatial difference as candidate antibodies;
5) Optionally calculating the humanness score of the engineered antibody sequence to further confirm that it is optimized in the direction of humanization. The humanness score is discussed e.g., Gao, S. H., et al. (2013). Monoclonal antibody humanness score and its applications. BMC Biotechnology, 13, which is incorporated herein by reference in its entirety.
6) Expression and purification of the selected engineered antibodies in vitro; and/or
7) Testing the binding affinity of the engineered antibody to the antigen, and selecting the engineered antibody whose affinity meets the actual needs.

### Providing a Non-Human Starting Antibody for Humanization

The methods of the present disclosure employ a non-human starting (e.g., donor) monoclonal antibody as starting material (“starting antibody”). Such monoclonal antibodies can be prepared using a wide variety of techniques known in the art including the use of hybridoma, recombinant, and phage display technologies, or a combination thereof (see, e.g., U.S. Pat. No. 9,550,986). For example, monoclonal antibodies can be produced using hybridoma techniques including those known in the art. The term “monoclonal antibody” as used herein is not limited to antibodies produced through hybridoma technology. The term “monoclonal antibody” refers to an antibody that is derived from a single clone, including any eukaryotic, prokaryotic, or phage clone.

Methods for producing and screening for specific antibodies using hybridoma technology are routine and well known in the art. In some embodiments, the present disclosure provides methods of generating monoclonal antibodies as well as antibodies produced by the method. The methods involve culturing a hybridoma cell secreting an antibody described herein wherein, preferably, the hybridoma is generated by fusing splenocytes isolated from a mouse or other rodent (e.g., a rat) immunized with an antigen of the interest with myeloma cells and then screening the hybridomas resulting from the fusion for hybridoma clones that secrete an antibody that can specifically bind a polypeptide described herein. Briefly, mice can be immunized with a desired target antigen. In some embodiments, the antigen is administered with an adjuvant to stimulate the immune response. Such adjuvants include complete or incomplete Freund's adjuvant, RIBI (muramyl dipeptides) or ISCOM (immunostimulating complexes). Such adjuvants can protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over e.g., 1, 2, 3, 4, or 5 weeks.

After immunization of an animal with a target antigen, antibodies and/or antibody-producing cells can be obtained from the animal. Antibody-containing serum is obtained from the animal by bleeding or sacrificing the animal. The serum can be used as it is obtained from the animal, an immunoglobulin fraction can be obtained from the serum, or the antibodies can be purified from the serum. Serum or immunoglobulins obtained in this manner are polyclonal, thus having a heterogeneous array of properties.

Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding the target antigen. Ascites fluid, which generally contains high levels of antibodies, can be generated by injecting positive hybridoma clones to the mice.

In some embodiments, hybridomas can be prepared from the immunized animal. After immunization, the animal is sacrificed and the splenic B cells are fused to immortalized myeloma cells as is well known in the art. In some embodiments, the myeloma cells do not secrete immunoglobulin polypeptides (a non-secretory cell line). After fusion and selection, the hybridomas are screened using the target antigen, or a portion thereof, or a cell expressing the target antigen. In some embodiments, the initial screening is performed using an enzyme-linked immunoassay (ELISA) or a radioimmunoassay (RIA), preferably an ELISA.

Hybridomas can be cultured and expanded in vivo in syngeneic animals, in animals that lack an immune system, e.g., nude mice, or in cell culture in vitro. Methods of selecting, cloning and expanding hybridomas are well known to those of ordinary skill in the art. In some embodiments, the hybridomas are mouse hybridomas. In some embodiments, the hybridomas are produced in a non-human, non-mouse species such as rats, sheep, pigs, goats, cattle or horses. In some embodiment, the hybridomas are human hybridomas, in which a human non-secretory myeloma is fused with a human cell expressing an antibody.

Antibody fragments that recognize specific epitopes can be generated by known techniques. For example, Fab and F(ab′)2 fragments described herein can be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

In some embodiments, the starting antibody is generated from single, isolated lymphocytes using a procedure referred to in the art as the selected lymphocyte antibody method (SLAM), as described in U.S. Pat. No. 5,627,052, PCT Publication WO 92/02551 and Babcock, J. S. et al. (1996) Proc. Natl. Acad. Sci. USA 93:7843-7848; each of which is incorporated herein by reference in its entirety. In this method, single cells secreting antibodies of interest, e.g., lymphocytes derived from any one of the immunized animals described herein, are screened using an antigen-specific hemolytic plaque assay, wherein the target antigen, or a subunit or a fragment thereof, is coupled to sheep red blood cells using a linker, such as biotin, and used to identify single cells that secrete antibodies with specificity for the target. Following identification of antibody-secreting cells of interest, heavy- and light-chain variable region cDNAs are rescued from the cells by reverse transcriptase-PCR and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to the target antigen. The amplified immunoglobulin sequences further can be manipulated in vitro, such as by in vitro affinity maturation methods such as those described in PCT Publication WO 97/29131 and PCT Publication WO 00/56772.

In vitro methods also can be used to provide a starting antibody. For example, an antibody library can be screened to identify an antibody having the desired binding specificity. The recombinant antibody library can be from a subject immunized with the target antigen, or a portion thereof, such as the extracellular domain. Alternatively, the recombinant antibody library can be from a naïve subject, i.e., one who has not been immunized with the target antigen, such as a human antibody library from a human subject who has not been immunized with the human antigen. Antibodies described herein are selected by screening the recombinant antibody library with the peptide comprising human antigen to thereby select those antibodies that recognize the target. To select engineered antibodies having a particular neutralizing activity, such as those with a particular an IC50, standard methods known in the art for assessing the inhibition of target activity may be used.

In certain embodiments, parental antibodies can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. In some embodiments, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria.

In some embodiments, the antibodies of the present disclosure can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine).

In some embodiments, the nucleic acid sequences encoding the variable domains of antibodies are sequences. In some embodiments, hybridoma sequencing is used. The methods involve sequencing of the variable regions of antibodies being produced from hybridoma cell lines. In some embodiments, total RNA is extracted from hybridoma cells and using PCR methods. The known non-variable flanking constant region sequences of respective antibody isotypes, the antibody variable regions (both heavy chain, VH, and light chain, VL) can be amplified for cloning and sequencing. In some embodiments, degenerate primers are used to amplify the variable regions of heavy (VH) and/or light (VL) chain antibody transcripts. In some embodiments, multiple cloned and sequenced VH and VL chains can be expressed in full-length antibody plasmid backbones, and resulting VH-VL pairs are examined for binding to the target antigen. In some embodiments, single-chain variable fragments (scFv) are synthesized.

### Structural Analysis of Antibodies

There are two general approaches to predicting the structure of a protein of interest: template-based modelling, in which the previously determined structure of a related protein is used to model the unknown structure of the target; and template-free modelling, which does not rely on global similarity to a structure in the Protein Data Bank (PDB) and hence can be applied to proteins with novel folds.

The steps in standard template-based modelling include selection of a suitable structural template; alignment of the target sequence to the template structure; and molecular modelling to account for mutations, insertions and deletions present in the target-template alignment. Closely related templates can be detected by using single-sequence search methods such as BLAST to scan the PDB sequences. To detect more distantly related templates, a target sequence profile built from a multiple-sequence alignment can be used to scan a database of sequence profiles for proteins of known structure by profile-profile comparison or can be matched to a library of structural templates to assess sequence-structure compatibility. Template selection methods return an initial target-template alignment that can be adjusted manually, often in an iterative manner after model building. Given an alignment to a template, established tools can be used to quickly construct molecular models of the target sequence by performing side-chain optimization only at mutated positions and by rebuilding the backbone around insertions and deletions. For target protein sequences that are only distantly related to proteins of known structure, more sophisticated approaches that rely on multiple templates and perform aggressive backbone conformational sampling may be required. Together with available crystal structures, template-based modelling approaches can provide structural information for roughly two-thirds of known protein families.

Template-free modelling approaches can be applied to proteins without global structural similarity to a protein in the PDB. Lacking a structural template, these methods require a conformational sampling strategy for generating candidate models, as well as a ranking criterion by which native-like conformations can be selected. The structure prediction process without a template typically begins with the construction of a multiple-sequence alignment of the target protein and related sequences. The sequences of the target and its homologues are then used to predict local structural features, such as secondary structure and backbone torsion angles, and non-local features, such as residue-residue contacts or inter-residue distances across the polypeptide chain. These predicted features guide the process of building 3D models of the target protein structure, which are then refined, ranked and compared with one another to select the final predictions. End-to-end deep neural network directly predicts the 3D coordinates of all heavy atoms for a given protein using embed multiple sequence alignments (MSAs) and pairwise features. Leveraging the insight that language models learn evolutionary patterns across millions of sequences, single-sequence protein structure prediction methods direct inference of structure from primary sequence using a large language model enables high resolution structure prediction.

In some embodiments, structural analysis of the antibody can be conducted to identify key framework residues in the starting framework regions that may be need to be retained if they are non-identical to those corresponding residue in the acceptor antibody. These key residues may be identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties) Three-dimensional immunoglobulin models are commonly available and are familiar to those skilled in the art. Computer programs are available which illustrate and display probable three-dimensional conformational structures of selected candidate immunoglobulin sequences. Inspection of the three-dimensional conformational structures permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved.

In certain embodiments, the antibody structure is predicted by deep learning-based folding methods, such as Alphafold2, RosettaFold, or VibrantFold.

In certain exemplary embodiments, the antibody structure is modeled using Antibody Modeler in Molecular Operating Environment (Molecular Operating Environment (MOE), 2011.10; Chemical Computing Group Inc., Montreal, QC, Canada). The MOE Antibody Homology Modeling accounts for the particular structural composition of antibodies when searching for template candidates and composing templates. As a result, models can be generated based on templates containing framework and CDR loops from different sources composed as dimers. In certain alternative embodiments, a knowledge-based approach can be applied with an underlying database of antibody structures currently in the Protein Data Bank (PDB), clustered by class, species, subclass and framework sequence identity. This database can be enriched with additional antibody structures and can be continually updated and reclustered.

In certain embodiments, multiple structural models can be provided for each starting antibody or each engineered antibody in order to generate a single consensus structure. The consensus structure is then used for further structure-based analysis. In other embodiments, structural models can be eliminated if they contain any deletion or gap in the modeled structure.

Having identified an appropriate structural model, one of ordinary skill in the art can annotate the modeled structure to identify CDRs or FRs by correlating the structure with the annotated sequence of the starting antibody provided above. For example, if there is a deletion or insertion at in the modeled structure, the structural model can be shifted or recalibrated to correlate with the structural positions of the original starting antibody.

Many programs are available for modeling and evaluating the 3D structure of antibodies (see, e.g., U.S. Pat. No. 7,117,096). For example, a molecular mechanics software may be employed for these purposes, examples of which include, but are not limited to CONGEN, SCWRL, UHBD, GENPOL and AMBER.

CONGEN (CONformation GENerator) is a program for performing conformational searches on segments of proteins (R. E. Bruccoleri (1993) Molecular Simulations 10, 151-174 (1993); R. E. Bruccoleri. E. Haber, J. Novotny, (1988) Nature 335, 564-568 (1988); R. Bruccoleri, M. Karplus. (1987) Biopolymers 26, 137-168). It is most suited to problems where one needs to construct undetermined loops or segments in a known structure, i.e. homology modeling. The basic energy function used includes terms for bonds, angles, torsional angels, improper angles, van der Waals and electrostatic interactions with distance dependent dielectric constant using Amber94 forcefield which can be determined using CONGEN. The program can be used to perform both conformational searches and structural evaluation using basic or refined scoring function. The program can calculate other properties of the molecules such as the solvent accessible surface area and conformational entropies, given steric constraints.

SCWRL is a side chain placing program that can be used to generate side chain rotamers and combinations of rotamers using the backbone dependent rotamer library (Dunbrack R L Jr, Karplus M (1993) J Mol Biol 230:543-574; Bower, M J, Cohen F E, Dunbrack R L (1997) J Mol Biol 267, 1268-1282). The library provides lists of chi1-chi2-chi3-chi4 values and their relative probabilities for residues at given phi-psi values. The program can further explore these conformations to minimize sidechain-backbone clashes and sidechain-sidechain clashes. Once the steric clash is minimized, the side chains and the backbone of the substituted segment can be energy minimized to relieve local strain using CONGEN (Bruccoleri and Karplus (1987) Biopolymers 26:137-168).

Several automatic programs that are developed specifically for building antibody structures may be used for structural modeling of antibody. The ABGEN program is an automated antibody structure generation algorithm for obtaining structural models of antibody fragments (Mandal et al. (1996) Nature Biotech. 14:323-328). ABGEN utilizes a homology based scaffolding technique and includes the use of invariant and strictly conserved residues, structural motifs of known Fab, canonical features of hypervariable loops, torsional constraints for residue replacements and key inter-residue interactions. Specifically, the ABGEN algorithm consists of two principal modules, ABalign and ABbuild. ABalign is the program that provides the alignment of an antibody sequence with all the V-region sequences of antibodies whose structures are known and computes alignment score scores. The highest scoring library sequence is considered to be the best fit to the test sequence. ABbuild then uses this best fit model output by ABalign to generate the three-dimensional structure and provides Cartesian coordinates for the desired antibody sequence.

WAM (Whitelegg N R J and Rees, A R (2000) Protein Engineering 13, 819-824) is an improved version of ABM which uses a combined algorithm (Martin, A C R, Cheetham, J C, and Rees A R (1989) PNAS 86, 9268-9272) to model the CDR conformations using the canonical conformations of CDRs loops from PDB database and loop conformations generated using CONGEN. In short, the modular nature of antibody structure makes it possible to model its structure using a combination of protein homology modeling and structure predictions.

In some embodiments, the following procedure can be used to model antibody structure. Because antibody is one of the most conserved proteins in both sequence and structure, homology models of antibodies are relatively straightforward, except for certain CDR loops that are not yet determined within existing canonical structures or those with insertion or deletions. In some cases, these loops can be modeled using algorithms that combine homology modeling with conformational search, or using deep learning algorithms as described herein.

### Construction of Engineered Antibodies Based on the Starting Antibody

The engineered antibodies can be made by introducing one or more mutations to a starting antibody sequence in silico. The mutations can be insertions, deletions, or substitutions. In some embodiments, it can involve at least or about 1, 2, 3, 4, 5, 6, 7, 8, 9, 10, 20, 30, 40, 50, 60, 70, 80, 90, or 100 amino acids. The insertions, deletions, and substitutions can be within the CDR sequence, or at one or both terminal ends of the CDR sequence, or can be with the framework region sequence, or at one or both terminal ends of the framework region sequence.

In some embodiments, the engineered antibody is a humanized antibody. Humanized antibodies can include antibodies having variable and constant regions derived from (or having the same amino acid sequence as those derived from) human germline immunoglobulin sequences of human immunoglobulin scaffold sequences. Humanized antibodies can include amino acid residues not encoded by human germline immunoglobulin sequences (e.g., mutations introduced by random or site-specific mutagenesis in vitro or by somatic mutation in vivo). Accordingly, humanized antibodies are chimeric antibodies wherein sequences from a non-human species are substituted by the corresponding human sequences. In some embodiments, one or more framework region sequences in the starting antibody are replaced by framework region sequences derived from (or having the same amino acid sequence as those derived from) human germline immunoglobulin sequences.

Potential human acceptor sequences for the CDRs of the starting antibody can be compiled from databases of human IG germline sequences or other human acceptor sequences. Known human Ig sequences are disclosed, e.g., the international ImMunoGeneTics information system (IMGT), and National Center for Biotechnology Information (NCBI), which are incorporated herein by reference in the entirety. In certain embodiments, the methods described herein employ a human germline sequence database compiled from publically available databases such as V base (http://vbase.mrc-cpe.cam.ac.uk/). In other embodiments, the germline databases described herein specifically exclude human germline sequences comprising free cysteine residues or human germline sequences that are missing conservative cysteine residues.

The framework includes the amino acid residues that are part of the variable region, but are not part of the CDRs (e.g., using the Kabat definition of CDRs). Therefore, a variable region framework is between about 100-120 amino acids in length but is intended to reference only those amino acids outside of the CDRs. In some embodiments, for the specific example of a heavy chain variable region and for the CDRs as defined by Kabat et al., framework region 1 corresponds to the domain of the variable region encompassing amino acids 1-30; region 2 corresponds to the domain of the variable region encompassing amino acids 36-49; region 3 corresponds to the domain of the variable region encompassing amino acids 66-94, and region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above.

In some embodiments, antibodies with desired properties (e.g., antigenicity, immunogenicity, immunomodulatory activity, expression of the antibody in a homologous host, expression of the antibody in a heterologous host, expression of the antibody in a plant cell, susceptibility of the antibody to in vitro post-translational modifications, and/or susceptibility of the antibody to in vivo post-translational modifications) are identified. In some embodiments, one or more amino acids or sequences in the starting antibody are replaced by the corresponding amino acids or sequences from the antibodies with desired properties.

In some embodiments, a library of engineered antibodies are prepared, e.g., by computers. Antibodies with desired properties can be selected from the library of engineered antibodies.

In some embodiments, engineered antibodies can be constructed based on the 3D structure of the starting antibody and then screened based on the spatial structure difference comparing to the starting antibody.

In some embodiments, the engineered antibodies are constructed to search in a protein database to find those segments that match in sequence pattern with the amino acid sequence of the region to be replaced, for example, framework region sequences of the starting antibody. Multiple suitable framework region sequences can be identified. In some embodiments, a conventional BLAST analysis may be employed to search for sequences with high homology to the framework region sequence.

Also optionally, single target sequence and/or multiple sequence alignment can be used to build a profile Hidden Markov Model (HMM). This HMM is then be used to search for both close and remote human homologues from a protein sequence database such as Kabat database of proteins and the human germline immunoglobulin database for frameworks. The Kabat database of proteins of immunological interest from various species can be used for designing diverse sequences for CDRs.

In some embodiments, potential human acceptor germline sequences for the CDRs of the starting antibody are compiled from databases of human IG germline sequences or other human acceptor germline sequences. Engineered antibodies are generated by mutating the amino acids in the framework region of the starting antibody based on the corresponding amino acids in the acceptor/germline antibodies. In some embodiments, the entire framework region of the starting antibody is replaced.

Each member of the engineered antibodies is grafted onto the corresponding region in the starting antibody (e.g., CDR) and tested for spatial structure difference in silico.

Using similar approaches, engineered antibodies can be constructed based on starting antibody from different regions of the starting antibody, such as CDR1, CDR2, CDR3 of the heavy chain and/or light chain, and tested for spatial structure difference. These engineered antibodies can be combined to allow simultaneous mutations to different regions of the starting antibody, thereby increasing the diversity of the engineered mutant antibodies.

All of the mutant antibody sequences selected in these processes are screened based on spatial structure difference comparing to the starting antibody.

In some embodiments, the methods involve generating a plurality of engineered antibodies (e.g., humanized antibodies). In some embodiments, these antibodies are generated by replacing, in silico, the framework sequences of the starting antibody with the framework sequences of antibodies with desired properties (e.g., human germline antibodies). The methods as described herein can provide a more efficient method for generating the engineered antibodies and determining the structure of the engineered antibodies. In some embodiments, at least 100, 200, 300, 400, 500, 600, 700, 800, 900, 1000, 10000, or 100000 engineered antibodies are generated and the structures are determined within a short time window (e.g., within 1, 2, 3, 4, 5, 6, or 7 days). In some embodiments, the methods as described herein can determine the structure of at least 100 engineered antibodies within 1 day and identify the engineered antibody with desired properties.

### Determining the Spatial Structure Difference Between the Engineered Antibody and the Starting Antibody

Engineered antibodies can be constructed based on the starting antibody and then screened based on the spatial structure difference between the engineered antibody and the starting antibody.

In some embodiments, engineered antibodies are generated in silico by making mutations to selected amino acids in the starting antibody.

In some embodiments, a target antibody is identified, and engineered antibodies are generated in silico by replacing one or more amino acids in the starting antibody (e.g., donor antibody) with the corresponding one or more amino acids in the target antibody (receptor antibody).

In some embodiment, the protein structure of the engineered antibodies can be determined. In some embodiments, a deep learning-based protein folding method, such as VibrantFold, AlphaFold2, or RoseTTaFold is used to predict the antibody structure of the newly generated engineered antibody sequences, and to obtain their respective three-dimensional protein structure models, including the structures of CDR loops. The three-dimensional structure of the variable regions of the engineered antibody can be compared against the three-dimensional structure of the original starting antibody, e.g., using the PyMOL software. In some embodiments, the three-dimensional structures from different programs are compared against each other, and the most reliable protein structure prediction is selected.

The deep learning-based folding algorithms methods have high-precision prediction of the three-dimensional structure of antibodies (including e.g., CDR loops). In some embodiments, the antibody structure prediction software is based on a deep learning protein folding algorithm (e.g., AlphaFold or AlphaFold2, trRosetta, or Vibrantfold platform).

In some embodiments, the protein structure of the engineered antibodies is determined using template-free structure prediction. In some embodiments, the protein structure of the engineered antibodies is determined ab initio. Prediction of the protein 3D structure, can be classified as template-based or template-free (which is also referred as “ab initio”). Homology modeling is template-base method by constructing an atomic-resolution model of the target protein from its amino acid sequence and an experimental three-dimensional structure of a related homologous protein (the “template”). The quality of the homology model is dependent on the quality of the sequence alignment and template structure. When there is no similar sequence in the PDB database, obtaining correct template becomes difficult. Deep learning-base method makes a major breakthrough in ab initio protein structure prediction, such as VibrantFold, AlphaFold2, or RoseTTaFold. Different from homology modeling, ab initio prediction is template-free, enables rapid solution of challenging X-ray crystallography and cryo-EM structure modeling problems, and provides insights into the functions of proteins of currently unknown structure.

AlphaFold or AlphaFold2 uses structures from the PDB to train a neural network to predict the distances between residues' C atoms (Senior, Andrew W., et al. “Improved protein structure prediction using potentials from deep learning.” Nature 577.7792 (2020): 706-710). After an initial prediction, the potential is minimized using a gradient-descent algorithm to achieve the most accurate predictions.

RoseTTaFold with a three-track network in which information at the 1D sequence level, the 2D distance map level, and the 3D coordinate level is successively transformed and integrated. The three-track network produces structure predictions with accuracies approaching those of DeepMind in CASP14. The methods are described e.g., in detail in M. Baek et al., “Accurate prediction of protein structures and interactions using a three-track neural network”, Science 10.1126/science.abj8754 (2021), which is incorporated herein by reference in its entirety.

Transform-restrained Rosetta (trRosetta) can be used to predict inter-residue orientations and distances uses a deep residual convolutional neural network. Transform-restrained Rosetta (trRosetta) uses the input sequence and a multiple sequence alignment in order to output predicted structural features, which are given to a Rosetta building protocol to come up with a final structure (Yang, Jianyi, et al. “Improved protein structure prediction using predicted interresidue orientations.” Proceedings of the National Academy of Sciences 117.3 (2020): 1496-1503). The network learns probability distributions from a PDB dataset, and extends this learning to orientation features (dihedral angles between residues). After high-resolution checks, a 30% sequence identity cut-off, and other requirements—such as sequence length and sequence homology—a total of 15,051 protein chains were collected and used for training.

VibrantFold platform is based on the Bumblebee deep learning framework. AlphaFold2, RoseTTaFold, or trRosetta rely on evolutionary information captured in multiple sequence alignments (MSAs), primarily on evolutionary couplings (co-evolution). Such information is not available for all proteins and is computationally expensive to generate. Whereas VibrantFold platform is alignment-free, using only single sequences as input which makes the prediction process highly efficient. The prediction of antibody structures by Vibrantfold can be completed within 1 minute on a conventional CPU-based machine for most cases. The structure prediction accuracy of Vibrantfold is comparable to Alphafold2 and Rosettafold.

In some embodiments, a database for the protein structure is provided. The methods involve querying an input amino acid sequence in the database by neural network. The neural network identifies the features that match best to the features of the proteins in the database, and construct the structure directly from the input amino acid. In this process, homology modeling is not required.

In some embodiments, the methods involve querying an input amino acid sequence in several databases of protein sequences, and constructing a multiple sequence alignment (MSA). This enables the determination of the parts of the sequence that are more likely to mutate, and provides correlations between them. In some embodiments, the methods further involve identifying proteins that may have a similar structure to the input (e.g., as templates), and constructing an initial representation of the structure. The multiple sequence alignment and the templates are then passed through a transformer. The transformer identifies what is more informative. The more relevant data are then used to construct a three-dimensional model of the structure. Unlike the traditional models, homology modeling is not used. In some embodiments, a static, final structure is directly generated in a single step.

In addition, the methods as described herein provide a computation efficient way to determine the structure of the protein (e.g., the structure of CDR loops). In some embodiments, the protein structure can be determined within 30, 40, 50, 60, 70, 80, 90, 100, 110, or 120 seconds.

The spatial structure difference between the engineered antibody and the starting antibody can be calculated based on one or more mathematical indicators including e.g., RMSD, template modeling (TM) score or local Distance Difference Test (IDDT) score.

In some embodiments, RMSD is calculated based on the following formula:

$$RMSD = \sqrt{\frac{{\sum }_{i = 1}^{N} {( {x}_{i} - {\hat{x}}_{i} )}^{2}}{N}}$$

wherein Xi refers to the position of an atom in the engineered antibody, \hat{x}ᵢ refers to the position of the corresponding atom in the starting antibody, thus (Xi-\hat{x}ᵢ) refers to the distance of the two atoms in their respective protein structures (e.g., relative position change in the engineered antibody); and N refers to the number of atoms.

In a preferred embodiment, RMSD is used to quantify the spatial structure difference between the engineered antibody and the starting antibody, e.g., for certain amino acids, for certain regions (such as CDRs, and FR), or for the entire antibody.

In some embodiments, RMSD is calculated for all non-H atoms in certain amino acids or certain regions (e.g., CDR loops). In some embodiments, RMSD is calculated for all atoms in certain amino acids or certain regions (e.g., CDR loops). In some embodiments, RMSD is calculated for all atoms in the peptide bonds. In some embodiments, RMSD is calculated for Ca atoms, or for all backbone atoms.

In some embodiments, the structural difference is measured based on the sum of heavy chain CDR1 RMSD, heavy chain CDR2 RMSD, Heavy chain CDR3 RMSD, light chain CDR1 RMSD, light chain CDR2 RMSD, and light chain CDR3 RMSD. In some embodiments, the structural difference is measured based on the sum of a subset of heavy chain CDR1 RMSD, heavy chain CDR2 RMSD, Heavy chain CDR3 RMSD, light chain CDR1 RMSD, light chain CDR2 RMSD, and light chain CDR3 RMSD. For example, in some embodiments, the structural difference is measured based on the sum of heavy chain CDR2 RMSD, Heavy chain CDR3 RMSD, light chain CDR2 RMSD, and light chain CDR3 RMSD.

In some embodiments, the structural difference is measured based on the sum of VHH CDR1 RMSD, VHH CDR2 RMSD, VHH CDR3 RMSD.

In some embodiments, an engineered antibody with a spatial structure difference (e.g., as measured by RMSD) that is below a threshold level are selected as candidate antibodies. In some embodiments, the threshold level is 1 Å, 1.5 Å, 2 Å, 2.5 Å, 3 Å, 3.5 Å, 4 Å, 4.5 Å, 5 Å, 5.5 Å, 6 Å, 6.5 Å or 7 Å.

The TM score is a measure of similarity between two protein structures. The TM-score is intended as a more accurate measure of the global similarity of full-length protein structures than the often used RMSD measure. TM-score has the value in (0,1], where 1 indicates a perfect match between two structures.

The TM-score between two protein structures (e.g., a template structure and a target structure) is defined by

$$TM - score = max [ \frac{1}{{L}_{target}} {\sum }_{i}^{{L}_{commnon}} \frac{1}{1 + {( \frac{{d}_{i}}{{d}_{0} ( {L}_{target} )} )}^{2}} ]$$

where L_{target} is the length of the amino acid sequence of the target protein that is interested, and L_{common} is the number of residues that commonly appear on the template and target structures. dᵢ is the distance between the ith pair of residues between the template and the target structures, and d₀(L_{target}) is a distance scale that normalizes distances. More details for TM-score can be found e.g., in Zhang et al, “Scoring function for automated assessment of protein structure template quality.” Proteins: Structure, Function, and Bioinformatics 57.4 (2004): 702-710; and Zhang et al, “TM-align: a protein structure alignment algorithm based on the TM-score.” Nucleic acids research 33.7 (2005): 2302-2309; both of which are incorporated herein by reference in the entirety. In some embodiments, an engineered antibody with a spatial structure difference (e.g., as measured by TM score) that is above a threshold level, e.g., 0.2, 0.3, 0.4, 0.5, 0.6, 0.7, 0.8, 0.9, or 0.95. In some embodiments, the TM score is calculated for one or more CDRs as described herein.

The IDDT score is a local superposition-free score for comparing protein structures and models using distance difference tests. It is a superposition-free score that evaluates local distance differences of all atoms in a model, including validation of stereochemical plausibility. The reference can be a single structure, or an ensemble of equivalent structures. IDDT measures how well the environment in a reference structure is reproduced in a protein model. It is computed over all pairs of atoms in the reference structure at a distance closer than a predefined threshold Ro (called inclusion radius), and not belonging to the same residue. These atom pairs define a set of local distances L. A distance is considered preserved in the model M if it is, within a certain tolerance threshold, the same as the corresponding distance in L. If one or both the atoms defining a distance in the set are not present in M, the distance is considered non-preserved. For a given threshold, the fraction of preserved distances is calculated. The final IDDT score is the average of four fractions computed using the thresholds 0.5 Å, 1 Å, 2 Å and 4 Å. The IDDT score can be computed using all atoms in the prediction (the default choice), but also using only distances between Ca atoms, or between backbone atoms. Interactions between adjacent residues can be excluded by specifying a minimum sequence separation parameter More details regarding IDDT score can be found e.g., in Mariani et al. “IDDT: a local superposition-free score for comparing protein structures and models using distance difference tests.” Bioinformatics 29.21 (2013): 2722-2728, which is incorporated herein by reference in its entirety. In some embodiments, an engineered antibody with a spatial structure difference (e.g., as measured by IDDT score) that is above a threshold level, e.g., 0.2, 0.3, 0.4, 0.5, 0.6, 0.7, 0.8, 0.9, 0.95, 0.98, or 0.99. In some embodiments, the IDDT score is calculated for one or more CDRs as described herein.

In some embodiments, a spatial structure difference (SSD) is calculated for all non-H atoms in certain amino acids or certain regions (e.g., CDR loops). In some embodiments, SSD is calculated for all atoms in certain amino acids or certain regions (e.g., CDR loops). In some embodiments, SSD is calculated for all atoms in the peptide bonds. In some embodiments, SSD is calculated for Ca atoms, or for all backbone atoms (e.g., for the entire protein or for the CDR loops).

In some embodiments, the selected candidate antibodies are synthesized and subject to further testing.

In some embodiments, the target antibody is a human antibody. In some embodiments, the starting antibody is a non-human antibody. In some embodiments, a humanness score is calculated for the VH domain and/or VL domain of the engineered antibody. In some embodiments, a humanness score is calculated for the VHH domain of the engineered antibody.

The humanness score is a measurement of how human-like the variable region of an antibody is. It has been determined that increased humanness score is correlated with decreased immunogenicity of antibodies. More details of humanness score can be found e.g., in Gao, S. H., et al. (2013). Monoclonal antibody humanness score and its applications. BMC Biotechnology, 13, which is incorporated herein by reference in its entirety. In some embodiments, the humanized antibody has a humanness score of about or greater than 40, 50, 60, 70, 80, or 90.

### Production of Engineered Antibodies

Engineered (e.g., humanized) antibodies can be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies described herein in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies described herein include Chinese Hamster Ovary (CHO cells), NSO myeloma cells, COS cells, SP2 cells, and HEK293 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. In some cases, it can be advantageous to transfect a host cell with DNA encoding functional fragments of either the light chain and/or the heavy chain of an antibody. Recombinant DNA technology can also be used to remove some, or all, of the DNA encoding either or both of the light and heavy chains that is not necessary for binding to the antigens of interest. The molecules expressed from such truncated DNA molecules are also encompassed by the antibodies described herein. In addition, bifunctional antibodies can be produced in which one heavy and one light chain are an antibody described herein and the other heavy and light chain are specific for an antigen other than the antigens of interest by crosslinking an antibody described herein to a second antibody by standard chemical crosslinking methods.

In certain systems for recombinant expression of an antibody, or antigen-binding portion thereof, a recombinant expression vector encoding both the antibody heavy chain and the antibody light chain is introduced into CHO cells by calcium phosphate-mediated transfection. Within the recombinant expression vector, the antibody heavy and light chain genes are each operatively linked to CMV promoter or AdMLP promoter regulatory elements to drive high levels of transcription of the genes. The recombinant expression vector can also carry selection markers, which allows for selection of CHO cells that have been transfected with the vector. Standard molecular biology techniques can be used to prepare the recombinant expression vector, transfect the host cells, select for transformants, culture the host cells and recover the antibody from the culture medium. Still further the disclosure provides a method of synthesizing a recombinant antibody described herein by culturing a host cell described herein in a suitable culture medium until a recombinant antibody described herein is synthesized. The method can further comprise isolating the recombinant antibody from the culture medium.

### Engineered Antibodies

In some embodiments, the engineered (e.g., humanized) antibodies as described herein, exhibit substantially similar biological activity, e.g., target binding affinity, as the parental non-human antibodies from which they are derived, e.g., as assessed by any one of several in vitro and in vivo assays known in the art. In certain preferred embodiments, the engineered antibody exhibits improved activity with respect to its corresponding parental antibody.

In some embodiments, the engineered antibody can inhibit the activity of the target antigen with an IC₅₀ of less than 1×10⁻⁵ M, 1×10⁻⁶ M, 1×10⁻⁷ M, 1×10⁻⁸ M, or 1×10⁻⁹ M. In some embodiments, the engineered antibody has an EC₅₀ of less than 1×10⁻⁵ M, 1×10⁻⁶ M, 1×10⁻⁷ M, 1×10⁻⁸ M, or 1×10⁻⁹ M.

In some implementations, the engineered antibody (or antigen-binding fragments thereof) specifically binds to the target protein with a dissociation rate (kdis) of less than 0.1 s⁻¹, less than 0.01 s⁻¹, less than 0.001 s⁻¹, less than 0.0001 s⁻¹, or less than 0.00001 s⁻¹. In some embodiments, kdis is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (ka) is greater than 1×10²/Ms, greater than 1×10³/Ms, greater than 1×10⁴/Ms, greater than 1×10⁵/Ms, or greater than 1×10⁶/Ms. In some embodiments, ka is less than 1×10⁵/Ms, less than 1×10⁶/Ms, or less than 1×10⁷/Ms.

Affinities can be deduced from the quotient of the kinetic rate constants (KD=kdis/ka). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., ELISA, RIA, Bio-Layer Interferometry (BLI) and surface plasmon resonance (SPR).

In some embodiments, the engineered antibodies at least have a comparable binding affinity. In some embodiments, the ratio of KD of the engineered antibodies with KD of the starting antibodies is less than 10, 5, 1, 0.1.

In some embodiments, the engineered antibodies or antigen-binding fragments thereof as described herein can increase immune response, activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds.

In some embodiments, the activity of the engineered antibodies is at least 50%, 60%, 70%, 80%, 90%, 100%, 2 folds, 3 folds, 5 folds, 10 folds, or 20 folds of the starting antibody.

In certain embodiments, the engineered antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region.

In certain embodiments, the engineered antibody comprises an engineered Fc region. Replacements of amino acid residues in the Fc portion to alter antibody effector function are known in the art. The Fc portion of an antibody mediates several important effector functions e.g. cytokine induction, ADCC, phagocytosis, complement dependent cytotoxicity (CDC) and half-life/clearance rate of antibody and antigen-antibody complexes.

In certain embodiments, the engineered antibody is derivatized or linked to another functional molecule (e.g., another peptide or protein). For example, a labeled binding protein of described herein can be derived by functionally linking an antibody or antibody portion described herein (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

Useful detectable agents with which an antibody may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody can also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

In some embodiments, the engineered antibody is further modified to generate glycosylation site mutants in which the O- or N-linked glycosylation site of the binding protein has been mutated.

In some embodiments, the glycosylation of the engineered antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation can increase the affinity of the antibody for antigen.

Additionally or alternatively, the engineered antibody can be further modified with an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery.

In some embodiments, the antibodies or antigen binding fragments do not have a functional Fc region. For example, the antibodies or antigen binding fragments are Fab, Fab′, F(ab′)2, and Fv fragments. In some embodiments, the Fc region has LALA mutations (L234A and L235A mutations in EU numbering), or LALA-PG mutations (L234A, L235A, P329G mutations in EU numbering).

The engineered antibodies can be tested for relevant biological activity and/or antibody properties. Determination of what constitutes a relevant antibody property is a case specific exercise. Non-limiting examples of antibody properties that can be relevant in some embodiments of the present disclosure include, but are not limited, to antigenicity, immunogenicity, immunomodulatory activity, expression of the antibody in a homologous host, expression of the antibody in a heterologous host, expression of the antibody in a plant cell, susceptibility of the antibody to in vitro post-translational modifications and susceptibility of the antibody to in vivo post-translational modifications. In some embodiments, the engineered antibodies have comparable or improved relevant biological activity and/or antibody properties as compared to the starting antibody.

The engineered antibodies described herein or fragments thereof can be assayed for immunospecific binding to a specific antigen and cross-reactivity with other antigens by any method known in the art Immunoassays that can be used to analyze immunospecific binding and cross-reactivity include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays.

Engineered antibodies described herein or fragments thereof can also be assayed for their ability to inhibit the binding of an antigen to its host cell receptor using techniques known to those of skill in the art. For example, cells expressing a receptor can be contacted with a ligand for that receptor in the presence or absence of an antibody or fragment thereof that is an antagonist of the ligand and the ability of the antibody or fragment thereof to inhibit the ligand's binding can measured by, for example, flow cytometry or a scintillation assay. The ligand or the antibody or antibody fragment can be labeled with a detectable compound such as a radioactive label (e.g., 32P, 35S, and 125I) or a fluorescent label (e.g., fluorescein isothiocyanate, rhodamine, phycoerythrin, phycocyanin, allophycocyanin, o-phthaldehyde and fluorescamine) to enable detection of an interaction between the ligand and its receptor. Alternatively, the ability of antibodies or fragments thereof to inhibit a ligand from binding to its receptor can be determined in cell-free assays.

A engineered antibody or a fragment thereof constructed and/or identified in accordance with the present disclosure can be tested in vitro and/or in vivo for its ability to modulate the biological activity of cells. Such ability can be assessed by, e.g., detecting the expression of antigens and genes; detecting the proliferation of cells; detecting the activation of signaling molecules (e.g., signal transduction factors and kinases); detecting the effector function of cells; or detecting the differentiation of cells. Techniques known to those of skill in the art can be used for measuring these activities. For example, cellular proliferation can be assayed by 3H-thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and FACS analysis. The activation of signaling molecules can be assayed, for example, by kinase assays and electrophoretic shift assays (EMSAs).

The engineered antibodies described herein, fragments thereof, or compositions thereof are preferably tested in vitro and then in vivo for the desired therapeutic or prophylactic activity prior to use in humans. For example, assays which can be used to determine whether administration of a specific pharmaceutical composition is indicated include cell culture assays in which a patient tissue sample is grown in culture and exposed to, or otherwise contacted with, a pharmaceutical composition, and the effect of such composition upon the tissue sample is observed. The tissue sample can be obtained by biopsy from the patient. This test allows the identification of the therapeutically most effective therapy (e.g., prophylactic or therapeutic agent) for each individual patient. In various specific embodiments, in vitro assays can be carried out with representative cells of cell types involved a particular disorder to determine if a pharmaceutical composition described herein has a desired effect upon such cell types. For example, in vitro assay can be carried out with cell lines.

In yet other forms of antibody assays, the effect of an antibody, a fragment thereof, or a composition described herein on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen which is conjugated to FITC or phycoerythrin, and measuring the number of T-cells by FACS.

The antibodies, fragments, or compositions described herein used to treat, manage, prevent, or ameliorate a viral infection or one or more symptoms thereof can be tested for their ability to inhibit viral replication or reduce viral load in in vitro assays. The antibodies or fragments thereof administered according to the methods described herein can also be assayed for their ability to inhibit or downregulate the expression of viral polypeptides. Techniques known to those of skill in the art, including, but not limited to, western blot analysis, northern blot analysis, and RT-PCR can be used to measure the expression of viral polypeptides.

The antibodies, compositions, or combination therapies described herein can be tested in suitable animal model systems prior to use in humans. Such animal model systems include, but are not limited to, rats, mice, chicken, cows, monkeys, pigs, dogs, rabbits, etc. Any animal system well-known in the art may be used. Several aspects of the procedure may vary; such aspects include, but are not limited to, the temporal regime of administering the therapies (e.g., prophylactic and/or therapeutic agents) whether such therapies are administered separately or as an admixture, and the frequency of administration of the therapies.

Animal models can be used to assess the efficacy of the antibodies, fragments thereof, or compositions described herein for treating, managing, preventing, or ameliorating a particular disorder or one or more symptom thereof.

The antibodies, fragments thereof of compositions described herein can be assayed for their ability to decrease the time course of a particular disorder by at least 25%, preferably at least 50%, at least 60%, at least 75%, at least 85%, at least 95%, or at least 99%. The antibodies, compositions, or combination therapies described herein can also be assayed for their ability to increase the survival period of organisms (e.g., humans) suffering from a particular disorder by at least 25%, preferably at least 50%, at least 60%, at least 75%, at least 85%, at least 95%, or at least 99%. Further, antibodies, fragments thereof, compositions, or combination therapies described herein can be assayed their ability reduce the hospitalization period of humans suffering from viral respiratory infection by at least 60%, preferably at least 75%, at least 85%, at least 95%, or at least 99%.

The toxicity and/or efficacy of the antibodies, fragments thereof, or compositions described herein can be assayed by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Antibodies that exhibit large therapeutic indices are preferred. While antibodies that exhibit toxic side effects can be used, care should be taken to design a delivery system that targets such agents to the site of affected tissue in order to minimize potential damage to uninfected cells and, thereby, reduce side effects.

Technological advances in the future may make it possible to measure in higher throughput properties that can currently be measured only in low throughput. One skilled in the art will readily see that the methods described herein may be used to correlate any antibody properties that are not easily measured with a high-throughput assay with other properties that are readily measured in high throughput. In some embodiments, high throughput screening can be used.

### Methods of Using the Engineered Antibodies

The engineered antibodies developed using the methods described herein can be used alone or in combination with other prophylactic or therapeutic agents for treating, managing, preventing or ameliorating a disorder or one or more symptoms thereof.

The present disclosure provides methods for preventing, managing, treating, or ameliorating a disorder comprising administering to a subject in need thereof one or more engineered antibodies described herein alone or in combination with one or more therapies (e.g., one or more prophylactic or therapeutic agents). The present disclosure also provides compositions comprising one or more antibodies described herein and methods of preventing, managing, treating, or ameliorating a disorder or one or more symptoms thereof utilizing said compositions. Additional therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

Any therapy that is known to be useful, or that has been used or is currently being used for the prevention, management, treatment, or amelioration of a disorder or one or more symptoms thereof can be used in combination with an antibody described herein.

The engineered antibodies described herein can be used directly against a particular antigen. In some embodiments, antibodies described herein belong to a subclass or isotype that is capable of mediating the lysis of cells to which the antibody binds. In a specific embodiment, the antibodies described herein belong to a subclass or isotype that, upon complexing with cell surface proteins, activates serum complement and/or mediates antibody dependent cellular cytotoxicity (ADCC) by activating effector cells such as natural killer cells or macrophages.

The ability of any particular antibody to mediate lysis of the target cell by complement activation and/or ADCC can be assayed. The cells of interest are grown and labeled in vitro; the antibody is added to the cell culture in combination with either serum complement or immune cells which may be activated by the antigen antibody complexes. Cytolysis of the target cells is detected by the release of label from the lysed cells. In fact, antibodies can be screened using the patient's own serum as a source of complement and/or immune cells. The antibody that is capable of activating complement or mediating ADCC in the in vitro test can then be used therapeutically in that particular patient.

The engineered antibodies described herein can also be used in diagnostic assays either in vivo or in vitro for detection/identification of the expression of an antigen in a subject or a biological sample (e.g., cells or tissues). Suitable diagnostic assays for the antigen and its antibodies depend on the particular antibody used. Non-limiting examples are an ELISA, sandwich assay, and steric inhibition assays. For in vivo diagnostic assays using the antibodies described herein, the antibodies can be conjugated to a label that can be detected by imaging techniques, such as X-ray, computed tomography (CT), ultrasound, or magnetic resonance imaging (MRI). The antibodies described herein can also be used for the affinity purification of the antigen from recombinant cell culture or natural sources.

### Systems, Software, and Interfaces

The methods described herein (e.g., predicting protein structure, calculating the spatial structure difference (e.g., RMSD), calculating the humanness score, and selecting candidate engineered antibodies) often require a computer, processor, software, module or other apparatus. Methods described herein typically are computer-implemented methods, and one or more portions of a method sometimes are performed by one or more processors. Embodiments pertaining to methods described herein generally are applicable to the same or related processes implemented by instructions in systems, apparatus and computer program products described herein. In some embodiments, processes and methods described herein are performed by automated methods In some embodiments, an automated method is embodied in software, modules, processors, peripherals and/or an apparatus comprising the like. As used herein, software refers to computer readable program instructions that, when executed by a processor, perform computer operations, as described herein.

In some embodiments, data or datasets can be characterized by one or more features or variables, including e.g., values for antibody sequences. In some embodiments, the sequencing apparatus is included as part of the system. In some embodiments, a system comprises a computing apparatus and a sequencing apparatus, where the sequencing apparatus is configured to receive physical nucleic acid and generate sequence reads, and the computing apparatus is configured to process the reads from the sequencing apparatus. The computing apparatus sometimes is configured to determine the starting antibody sequence from the sequence reads.

Implementations of the subject matter and the functional operations described herein can be implemented in digital electronic circuitry, in tangibly-embodied computer software or firmware, in computer hardware, including the structures described herein and their structural equivalents, or in combinations of one or more of the structures. Implementations of the subject matter described herein can be implemented as one or more computer programs, i.e., one or more modules of computer program instructions encoded on a tangible program carrier for execution by, or to control the operation of, a processing device. Alternatively, or in addition, the program instructions can be encoded on a propagated signal that is an artificially generated signal, e.g., a machine-generated electrical, optical, or electromagnetic signal that is generated to encode information for transmission to suitable receiver apparatus for execution by a processing device. A machine-readable medium can be a machine-readable storage device, a machine-readable storage substrate, a random or serial access memory device, or a combination of one or more of them.

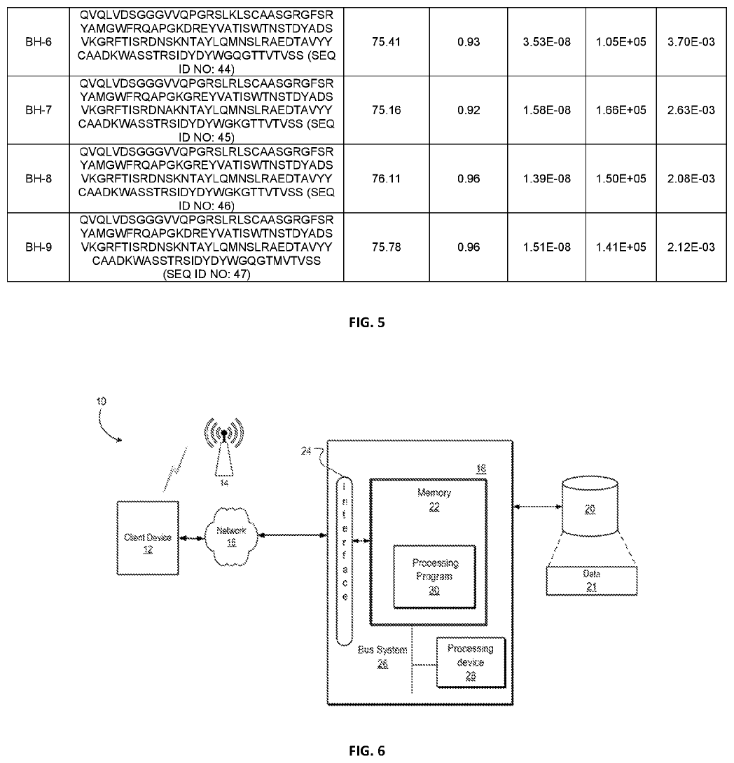

Referring to FIG. 6, system 10 processes data via applying a processor to the input data, and outputs information (e.g., predicting protein structure, calculating the spatial structure difference (e.g., RMSD), calculating the humanness score, and selecting candidate engineered antibodies). System 10 includes client device 12, data processing system 18, data repository 20, network 16, and wireless device 14. The processor processes the input data based on the methods described herein.

Data processing system 18 retrieves, from data repository 20, data 21 representing one or more values for the processor parameter, including e.g., the RMSD threshold level and/or the targeting antibody sequences. Data processing system 18 inputs the retrieved data into a processor, e.g., into data processing program 30. In this embodiment, data processing program 30 is programmed to introduce mutations to starting antibody sequences, predict protein structure, calculate the spatial structure difference (e.g., RMSD), calculate the humanness score, and select candidate engineered antibodies.

Data processing system 18 generates data for a graphical user interface that, when rendered on a display device of client device 12, display a visual representation of the output. In some embodiments, the values for these parameters can be stored in data repository 20 or memory 22.

Client device 12 can be any sort of computing device capable of taking input from a user and communicating over network 16 with data processing system 18 and/or with other client devices. Client device 12 can be a mobile device, a desktop computer, a laptop computer, a cell phone, a personal digital assistant (PDA), a server, an embedded computing system, and so forth.

Data processing system 18 can be any of a variety of computing devices capable of receiving data and running one or more services. In some embodiments, data processing system 18 can include a server, a distributed computing system, a desktop computer, a laptop computer, a cell phone, and the like. Data processing system 18 can be a single server or a group of servers that are at a same position or at different positions (i.e., locations). Data processing system 18 and client device 12 can run programs having a client-server relationship to each other. Although distinct modules are shown in the figure, in some embodiments, client and server programs can run on the same device.

Data processing system 18 can receive data from wireless device 14 and/or client device 12 through input/output (I/O) interface 24 and data repository 20. Data repository 20 can store a variety of data values for data processing program 30. The processing program (which may also be referred to as a program, software, a software application, a script, or code) can be written in any form of programming language, including compiled or interpreted languages, or declarative or procedural languages, and it can be deployed in any form, including as a stand-alone program or as a module, component, subroutine, or other unit suitable for use in a computing environment. The data processing program may, but need not, correspond to a file in a file system. The program can be stored in a portion of a file that holds other programs or information (e.g., one or more scripts stored in a markup language document), in a single file dedicated to the program in question, or in multiple coordinated files (e.g., files that store one or more modules, sub programs, or portions of code). The data processing program can be deployed to be executed on one computer or on multiple computers that are located at one site or distributed across multiple sites and interconnected by a communication network.

Interface 24 can be a type of interface capable of receiving data over a network, including, e.g., an Ethernet interface, a wireless networking interface, a fiber-optic networking interface, a modem, and so forth. Data processing system 18 also includes a processing device 28. As used herein, a “processing device” encompasses all kinds of apparatuses, devices, and machines for processing information, such as a programmable processor, a computer, or multiple processors or computers. The apparatus can include special purpose logic circuitry, e.g., an FPGA (field programmable gate array) or an ASIC (application specific integrated circuit) or RISC (reduced instruction set circuit). The apparatus can also include, in addition to hardware, code that creates an execution environment for the computer program in question, e.g., code that constitutes processor firmware, a protocol stack, an information base management system, an operating system, or a combination of one or more of them.

Data processing system 18 also includes a memory 22 and a bus system 26, including, for example, a data bus and a motherboard, which can be used to establish and to control data communication between the components of data processing system 18. Processing device 28 can include one or more microprocessors. Generally, processing device 28 can include an appropriate processor and/or logic that is capable of receiving and storing data, and of communicating over a network. Memory 22 can include a hard drive and a random access memory storage device, including, e.g., a dynamic random access memory, or other types of non-transitory, machine-readable storage devices. Memory 22 stores data processing program 30 that is executable by processing device 28. These computer programs may include a data engine for implementing the operations and/or the techniques described herein. The data engine can be implemented in software running on a computer device, hardware or a combination of software and hardware.

Various methods and formulae can be implemented, in the form of computer program instructions, and executed by a processing device. Suitable programming languages for expressing the program instructions include, but are not limited to, Python, C, C++, an embodiment of FORTRAN such as FORTRAN77 or FORTRAN90, Java, Visual Basic, Perl, Tcl/Tk, JavaScript, ADA, and statistical analysis software, such as SAS, R, MATLAB, SPSS, and Stata etc. Various aspects of the methods may be written in different computing languages from one another, and the various aspects are caused to communicate with one another by appropriate system-level-tools available on a given system.

The processes and logic flows described in this disclosure can be performed by one or more programmable computers executing one or more computer programs to perform functions by operating on input information and generating output. The processes and logic flows can also be performed by, and apparatus can also be implemented as, special purpose logic circuitry, e.g., an FPGA (field programmable gate array) or an ASIC (application specific integrated circuit) or RISC.

Computers suitable for the execution of a computer program include, by way of example, general or special purpose microprocessors, or both, or any other kind of central processing unit.

Computer readable media suitable for storing computer program instructions and information include various forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and (Blue Ray) DVD-ROM disks. The processor and the memory can be supplemented by, or incorporated in, special purpose logic circuitry.

To provide for interaction with a user, implementations of the subject matter described in this disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. In addition, a computer can interact with a user by sending documents to and receiving documents from a device that is used by the user; for example, by sending web pages to a web browser on a user's client device in response to requests received from the web browser.

Implementations of the subject matter described herein can be implemented in a computing system that includes a back end component, e.g., as an information server, or that includes a middleware component, e.g., an application server, or that includes a front end component, e.g., a client computer having a graphical user interface or a Web browser through which a user can interact with an implementation of the subject matter, or any combination of one or more such back end, middleware, or front end components. The components of the system can be interconnected by any form or medium of digital information communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, the server can be in the cloud via cloud computing services.

While this disclosure includes many specific implementation details, these should not be construed as limitations on the scope of any of what may be claimed, but rather as descriptions of features that may be specific to particular implementations. Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are described in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the claims. For example, the actions recited in the claims can be performed in a different order and still achieve desirable results. In one embodiment, the processes depicted in the accompanying figures do not necessarily require the particular order shown, or sequential order, to achieve desirable results. In some implementations, multitasking and parallel processing may be advantageous.

### EXAMPLES

The invention is further described in the following examples, which do not limit the scope of the invention described in the claims.

### Example 1. Humanization of Mouse Antibody A

The murine antibody A against antigen M consists of two heavy chains and two light chains. More details can be found e.g., in Chowdhury, P. S., et al. (1998). Isolation of a high-affinity stable single-chain Fv specific for mesothelin from DNA-immunized mice by phage display and construction of a recombinant immunotoxin with anti-tumor activity. Proceedings of the National Academy of Sciences, 95 (2), 669-674, which is incorporated herein by reference in its entirety.

In order to humanize antibody A, the gene sequences of the CDR regions of the heavy chain and light chain of antibody A were obtained. While keeping the CDR gene sequence of antibody A, the heavy chain and light chain framework sequences of antibody A were replaced by with pre-collected human germline antibody framework sequences.

A deep learning-based protein structure prediction software (such as VibrantFold/AlphaFold2/RoseTTaFold) was used to predict the antibody structure of the newly generated engineered antibodies, and to obtain their respective three-dimensional protein structure models.

PyMOL, a program for molecular visualization, was used to compare the three-dimensional structure of the variable regions of the engineered antibodies with the three-dimensional structure of the original mouse antibody.

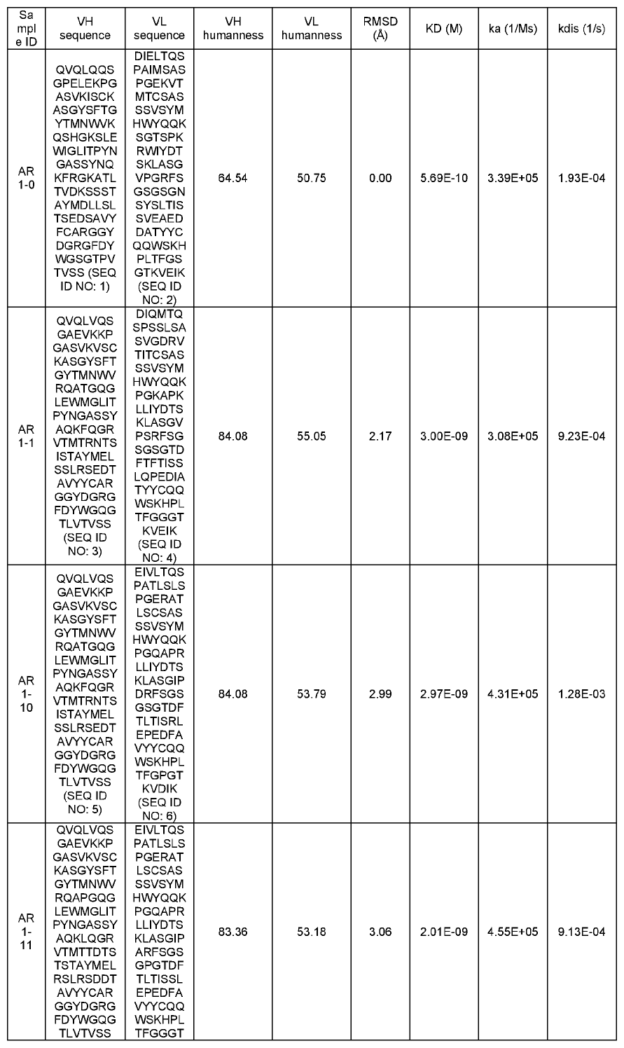

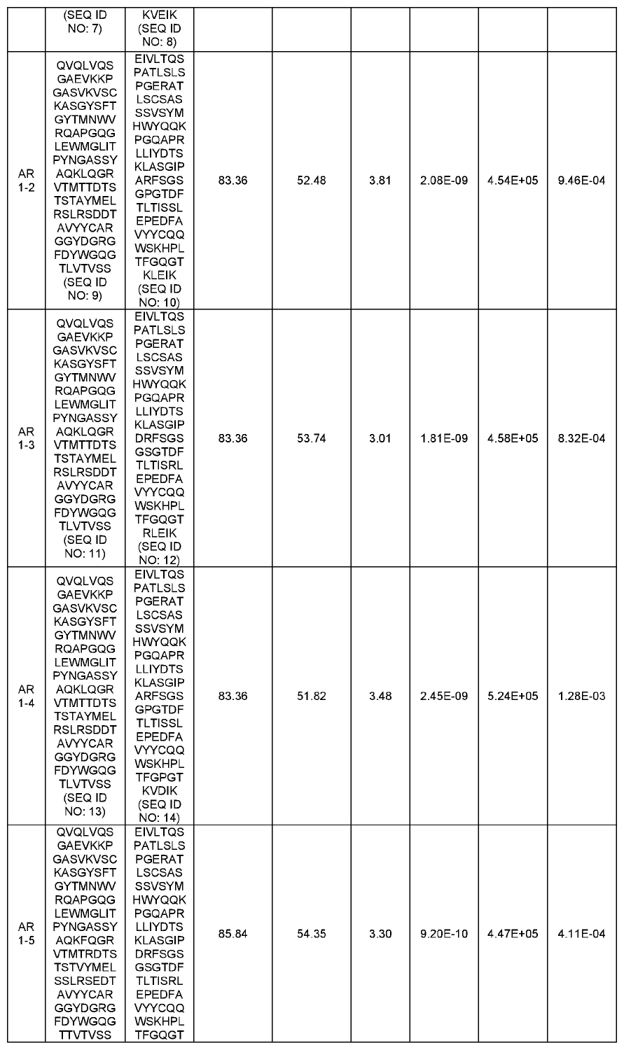

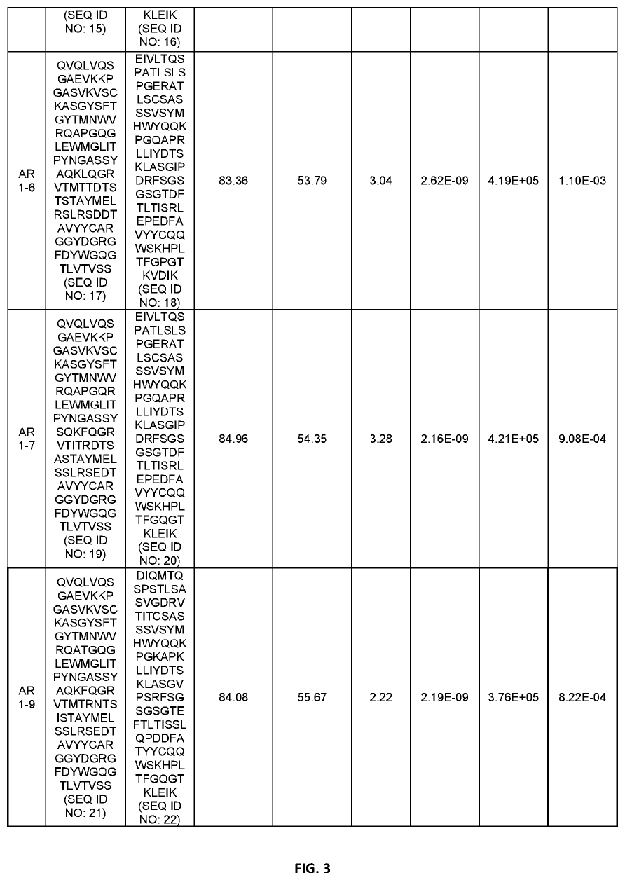

RMSD was used to quantify the spatial structure gap between the starting antibody and each of the engineered antibodies. RMSD was calculated based on the sum of the below RMSD values: the heavy chain CDR 1 RMSD, the heavy chain CDR2 RMSD, the heavy chain CDR3 RMSD, the light chain CDR 1 RMSD, the light chain CDR2 RMSD, and the light chain CDR3 RMSD (FIG. 3).

A humanness score was calculated for the heavy chain and light chain of the starting antibody (antibody A) and the heavy chain and light chain of the engineered antibodies based on the method described in the below reference: Gao, S. H., et al. (2013). Monoclonal antibody humanness score and its applications. BMC Biotechnology, 13.

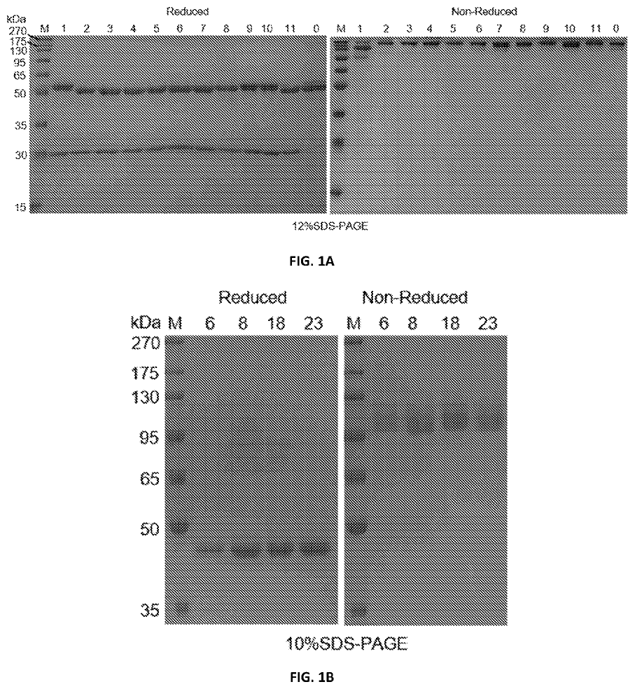

The engineered antibody with a RMSD value below a threshold value were picked as the candidate antibodies. Subsequent experimental verification of candidate antibodies involved: synthesizing corresponding DNA fragments according to the candidate sequences, loading the DNA fragments into vector plasmids, and introducing them into host cells for in vitro expression. The engineered antibodies expressed by the cells were separated and purified (FIGS. 1A-1B).

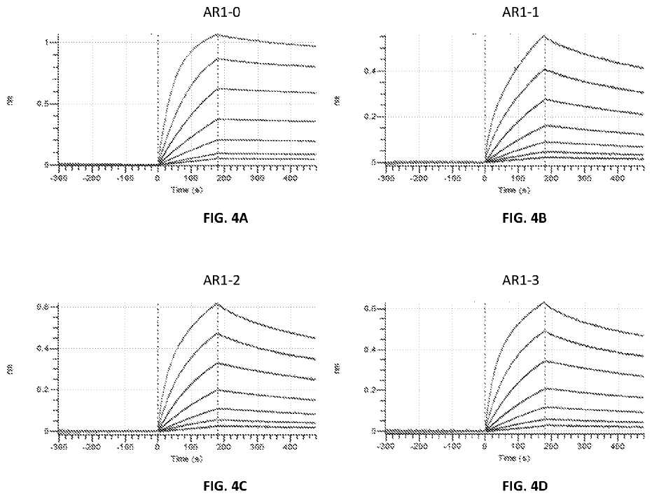

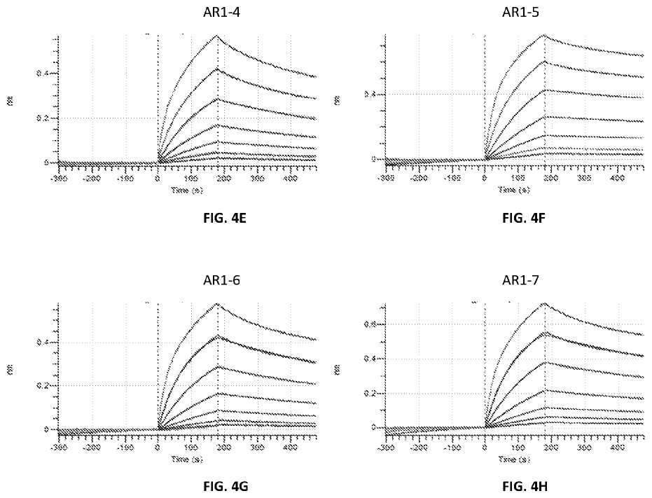

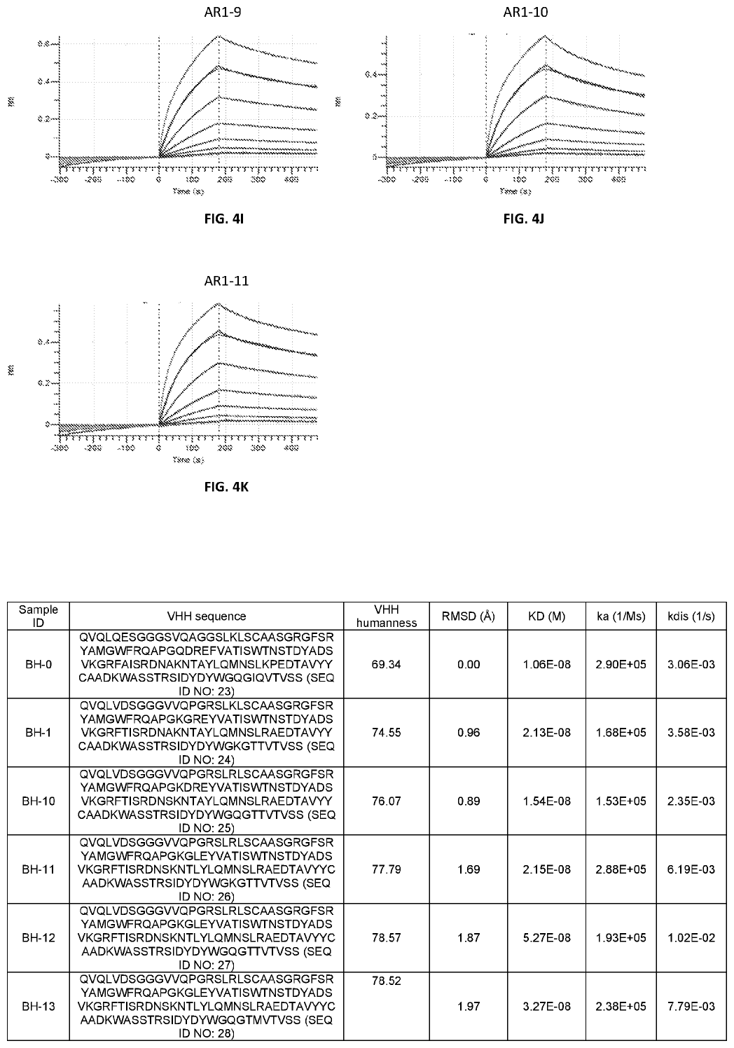

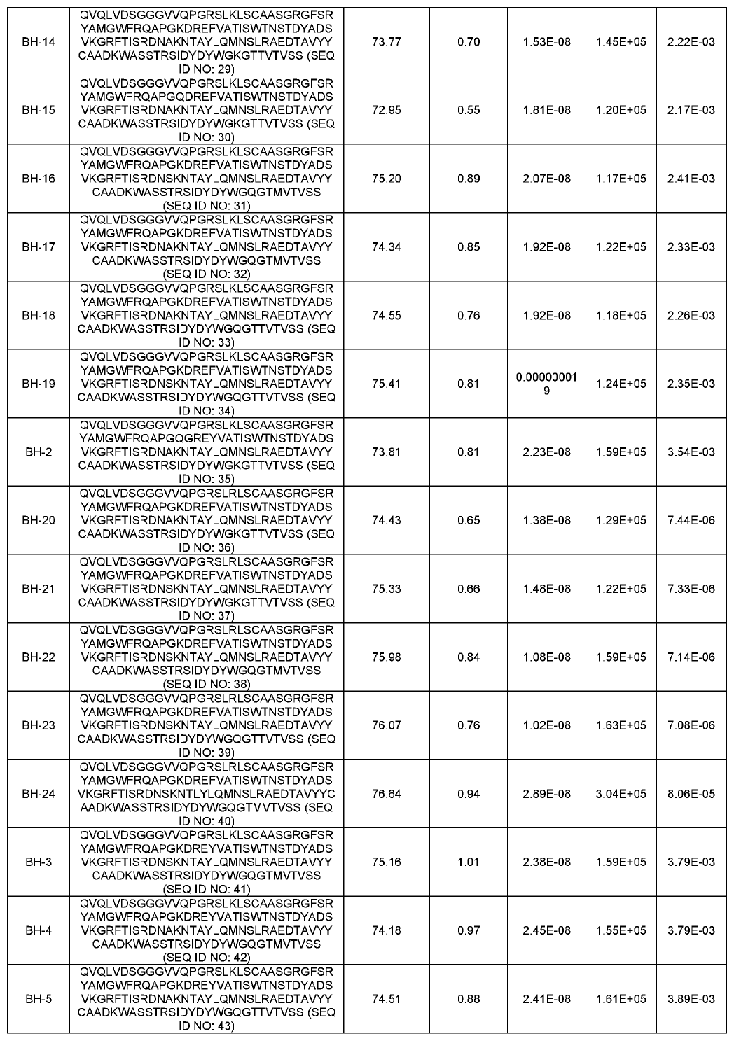

BLI was used to test the binding affinity between the purified engineered antibodies and antigen M, and to select the engineered antibodies whose affinity meets actual needs (FIG. 3 and FIG. 4).

### Example 2. Humanization of Camel-Derived Antibody B

Camel-derived antibody B against antigen Nis a single domain antibody composed of only heavy chains. More details can be found e.g., in WO2007042289A2, which is incorporated herein by reference in its entirety.

In order to humanize antibody B, the cells secreting antibody B were sequenced to obtain the heavy chain CDR sequence of antibody B. While keeping the CDR sequence of antibody B, the amino acids in the heavy chain framework sequence of antibody B was replaced with pre-collected human antibody germline framework sequences in the computer one by one. In order to increase antibody diversity, virtual point mutations are also introduced.

Deep learning-based protein structure prediction software (such as VibrantFold/AlphaFold2/RoseTTaFold) was used to predict the antibody structure of the newly generated engineered antibody sequences, and to obtain their respective three-dimensional protein structure models.

PyMOL was used to compare the three-dimensional structure of the engineered antibody variable region with the three-dimensional structure of the original camel-derived variable region antibody.

RMSD was used to quantify the structural gap between the starting antibody and each of the engineered antibodies.

RMSD was calculated based on the sum of the below RMSD values: the heavy chain CDR 1 RMSD, the heavy chain CDR2 RMSD, and the heavy chain CDR3 RMSD. The engineered antibody with a RMSD value below a threshold value were picked as the candidate antibodies.

A humanness score was calculated for the heavy chain of the starting antibody (antibody B) and the heavy chain of the engineered antibodies based on the method described in the below reference: Gao, S. H., et al. (2013). Monoclonal antibody humanness score and its applications. BMC Biotechnology, 13.

The candidate antibodies were subsequently verified by affinity experiments. Finally, an anti-N humanized engineered antibody that meets the affinity requirements was obtained (FIG. 5).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A computer-implemented method for producing an engineered antibody, the method comprising:
(a) generating, in silico, an engineered antibody by introducing one or more mutations to a starting antibody sequence;
(b) determining a spatial structure difference (SSD) in CDR between the engineered antibody and the starting antibody;
(c) determining the engineered antibody having a spatial structure difference that meets a threshold level; and
(d) selecting the engineered antibody.

2. The method of claim 1, wherein the spatial structure difference is predicted by a template free protein folding method.

3. The method of claim 1 or 2, wherein the method comprises querying an input amino acid sequence of the engineered antibody in a database by neural network; and constructing a protein structure directly from the neural network.

4. The method of any one of claims 1-3, wherein the SSD is measured by Root Mean Squared Deviation (RMSD), Template Modeling (TM) score, or IDDT score.

5. The method of claim 4, wherein the SSD is measured by RMSD.

6. The method of claim 5, wherein the RMSD is within 1 Å.

7. The method of claim 5, wherein the RMSD is within 2 Å.

8. The method of claim 5, wherein the RMSD is within 3 Å.

9. The method of claim 5, wherein the RMSD is within 4.5 Å.

10. The method of any one of claims 1-9, wherein the spatial structure difference is selected from heavy chain CDR1SSD, the heavy chain CDR2 SSD, the heavy chain CDR3 SSD, the light chain CDR 1 SSD, the light chain CDR2 SSD, and the light chain CDR3 SSD.

11. The method of any one of claims 1-9, wherein the spatial structure difference is calculated by combining two or more SSD values selected from: the heavy chain CDR1 SSD, the heavy chain CDR2 SSD, the heavy chain CDR3 SSD, the light chain CDR1 SSD, the light chain CDR2 SSD, and the light chain CDR3 SSD.

12. The method of any one of claims 1-9, wherein the spatial structure difference is the sum of the heavy chain CDR1 SSD, the heavy chain CDR2 SSD, the heavy chain CDR3 SSD, the light chain CDR 1 SSD, the light chain CDR2 SSD, and the light chain CDR3 SSD.

13. The method of any one of claims 1-9, wherein the spatial structure difference is the sum of the heavy chain CDR1 SSD, the heavy chain CDR2 SSD, and the heavy chain CDR3 SSD.

14. The method of any one of claims 1-13, wherein the mutations are (a) point mutations or (b) replacing a part of the sequence of the starting antibody.

15. The method of claim 14, wherein framework sequences of the starting antibody are replaced by framework sequences of a human antibody.

16. The method of any one of claims 1-15, wherein the engineered antibody is a humanized antibody.

17. The method of any one of claims 1-16, wherein the engineered antibody has one or more improved antibody characteristics as comparing to the starting antibody.

18. The method of any one of claims 1-17, wherein the engineered antibody comprises a sequence of an antibody that is selected from: AR1-1, AR1-2, AR1-3, AR1-4, AR1-5, AR1-6, AR1-7, AR1-9, AR1-10, AR1-11, BH-1, BH-2, BH-3, BH-4, BH-5, BH-6, BH-7, BH-8, BH-9, BH-10, BH-11, BH-12, BH-13, BH-14, BH-15, BH-16, BH-17, BH-18, BH-19, BH-20, BH-21, BH-22, BH-23, and BH-24.

19. The method of any one of claims 1-18, wherein the method further comprises producing the engineered antibody.

20. A method of producing a humanized antibody, the method comprising:
(a) identifying CDR sequences and framework sequences of a non-human starting antibody;
(b) generating a plurality of humanized antibodies by replacing, in silico, the framework sequences of the starting antibody with the framework sequences of human germline antibodies;
(c) determining the spatial structure differences (SSD) between the plurality of humanized antibodies and the starting antibody; and
(d) selecting, a humanized antibody from the plurality of humanized antibodies with a spatial structure difference that meets a threshold value.

21. The method of claim 20, wherein the method comprises querying an input amino acid sequence of the plurality of humanized antibodies in a database by neural network; and
constructing a protein structure directly from the neural network.

22. The method of claim 20 or 21, wherein the SSD is measured by RMSD.

23. The method of claim 22, wherein the RMSD is within 1 Å.

24. The method of claim 22, wherein the RMSD is within 2 Å.

25. The method of claim 22, wherein the RMSD is within 3 Å.

26. The method of claim 22, wherein the RMSD is within 4.5 Å.

27. The method of any one of claims 20-26, wherein the spatial structure differences are calculated for each atom in the CDR sequences.

28. The method of any one of claims 20-27, wherein the spatial structure differences are calculated for each non-H atom in the CDR sequences.

29. The method of any one of claims 20-28, wherein the spatial structure differences are calculated for each atom in peptide bonds in the CDR sequences.

30. The method of any one of claims 20-29, wherein the selected humanized antibody comprises a VHH.

31. The method of claim 30, wherein the spatial structure differences are calculated by combining VHH CDR1 SSD, VHH CDR2 SSD, and VHH CDR3 SSD.

32. The method of claim 30, wherein the spatial structure differences are calculated by adding one or more of VHH CDR1 SSD, VHH CDR2 SSD, and VHH CDR3 SSD.

33. The method of any one of claims 20-29, wherein the selected humanized antibody comprises a VH and a VL.

34. The method of claim 33, wherein the spatial structure differences are calculated by combining the heavy chain CDR1 SSD, the heavy chain CDR2 SSD, the heavy chain CDR3 SSD, the light chain CDR 1 SSD, the light chain CDR2 SSD, and the light chain CDR3 SSD.

35. The method of any one of claims 20-34, wherein the method further comprises introducing one or more mutations to the plurality of humanized antibodies.

36. The method of any one of claims 20-35, wherein the binding affinity of the selected humanized antibody (KD) is less than 1×10⁻⁸ M.

37. The method of any one of claims 20-36, wherein at least 100 or 1000 humanized antibodies are generated.

38. The method of any one of claims 20-37, wherein the SSD between one humanized antibody and the starting antibody is determined within 1 minute.

39. The method of any one of claims 20-38, wherein the method is completed within 1 day.

40. The method of any one of claims 20-39, wherein the method further comprises making a vector comprising a nucleic acid sequence encoding the selected humanized antibody; and expressing the selected humanized antibody.

41. The method of any one of claims 20-40, wherein the selected humanized antibody comprises a human constant region.

42. One or more machine-readable hardware storage devices for storing instructions that are executable by one or more data processing devices to perform the method of any one of claims 1-18 and 20-39.

43. A system comprising:
one or more data processing devices; and
one or more machine-readable hardware storage devices that store instructions that are executable by the one or more data processing devices to execute the method of any one of claims 1-41.

44. The system of claim 43, wherein the system further comprises one or more devices for making and expressing nucleic acid sequences.

45. A method for producing an engineered antibody, the method comprising:
(a) providing an amino acid sequence of an engineered antibody, wherein the engineered antibody is generated by the method comprising the following steps:
(i) generating, in silico, an engineered antibody by introducing one or more mutations to a starting antibody sequence;
(ii) determining a spatial structure difference (SSD) in CDR between the engineered antibody and the starting antibody, wherein the spatial structure is predicted by a template free protein folding method;
(iii) determining the engineered antibody having a spatial structure difference that meets a threshold level; and
(b) producing the engineered antibody.

46. The method of claim 45, wherein the spatial structure difference is predicted by a template free protein folding method.

47. The method of claim 45 or 46, wherein the method comprises querying an input amino acid sequence of the engineered antibody in a database by neural network; and
constructing a protein structure directly from the neural network.

48. The method of any one of claims 45-47, wherein the SSD is measured by RMSD, TM score, or IDDT score.

49. The method of any one of claims 45-48, wherein the mutations are a) point mutations or b) replacing a part of the sequence of the starting antibody.

50. The method of claim 49, wherein framework sequences of the starting antibody are replaced by framework sequences of a human antibody.


SEQUENCE LISTING (47 sequences)

SEQ ID NO: 1 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 2 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 3 - protein, 119 aa, synthetic construct
QVQLVQSGAEVKKPGASVKVSCKASGYSFTGYTMNWVRQATGQGLEWMGLITPYNGASSYAQKFQGRVTMTRNTSISTAYMELSSLRSEDTAVYYCARGGYDGRGFDYWGQGTLVTVSS

SEQ ID NO: 4 - protein, 106 aa, synthetic construct
DIQMTQSPSSLSASVGDRVTITCSASSSVSYMHWYQQKPGKAPKLLIYDTSKLASGVPSRFSGSGSGTDFTFTISSLQPEDIATYYCQQWSKHPLTFGGGTKVEIK

SEQ ID NO: 5 - protein, 119 aa, synthetic construct; identical to SEQ ID NO: 3

SEQ ID NO: 6 - protein, 106 aa, synthetic construct
EIVLTQSPATLSLSPGERATLSCSASSSVSYMHWYQQKPGQAPRLLIYDTSKLASGIPDRFSGSGSGTDFTLTISRLEPEDFAVYYCQQWSKHPLTFGPGTKVDIK

SEQ ID NO: 7 - protein, 119 aa, synthetic construct
QVQLVQSGAEVKKPGASVKVSCKASGYSFTGYTMNWVRQAPGQGLEWMGLITPYNGASSYAQKLQGRVTMTTDTSTSTAYMELRSLRSDDTAVYYCARGGYDGRGFDYWGQGTLVTVSS

SEQ ID NO: 8 - protein, 106 aa, synthetic construct
EIVLTQSPATLSLSPGERATLSCSASSSVSYMHWYQQKPGQAPRLLIYDTSKLASGIPARFSGSGPGTDFTLTISSLEPEDFAVYYCQQWSKHPLTFGGGTKVEIK

SEQ ID NO: 9 - protein, 119 aa, synthetic construct; identical to SEQ ID NO: 7

SEQ ID NO: 10 - protein, 106 aa, synthetic construct
EIVLTQSPATLSLSPGERATLSCSASSSVSYMHWYQQKPGQAPRLLIYDTSKLASGIPARFSGSGPGTDFTLTISSLEPEDFAVYYCQQWSKHPLTFGQGTKLEIK

SEQ ID NO: 11 - protein, 119 aa, synthetic construct; identical to SEQ ID NO: 7

SEQ ID NO: 12 - protein, 106 aa, synthetic construct
EIVLTQSPATLSLSPGERATLSCSASSSVSYMHWYQQKPGQAPRLLIYDTSKLASGIPDRFSGSGSGTDFTLTISRLEPEDFAVYYCQQWSKHPLTFGQGTRLEIK

SEQ ID NO: 13 - protein, 119 aa, synthetic construct; identical to SEQ ID NO: 7

SEQ ID NO: 14 - protein, 106 aa, synthetic construct
EIVLTQSPATLSLSPGERATLSCSASSSVSYMHWYQQKPGQAPRLLIYDTSKLASGIPARFSGSGPGTDFTLTISSLEPEDFAVYYCQQWSKHPLTFGPGTKVDIK

SEQ ID NO: 15 - protein, 119 aa, synthetic construct
QVQLVQSGAEVKKPGASVKVSCKASGYSFTGYTMNWVRQAPGQGLEWMGLITPYNGASSYAQKFQGRVTMTRDTSTSTVYMELSSLRSEDTAVYYCARGGYDGRGFDYWGQGTTVTVSS

SEQ ID NO: 16 - protein, 106 aa, synthetic construct
EIVLTQSPATLSLSPGERATLSCSASSSVSYMHWYQQKPGQAPRLLIYDTSKLASGIPDRFSGSGSGTDFTLTISRLEPEDFAVYYCQQWSKHPLTFGQGTKLEIK

SEQ ID NO: 17 - protein, 119 aa, synthetic construct; identical to SEQ ID NO: 7

SEQ ID NO: 18 - protein, 106 aa, synthetic construct; identical to SEQ ID NO: 6

SEQ ID NO: 19 - protein, 119 aa, synthetic construct
QVQLVQSGAEVKKPGASVKVSCKASGYSFTGYTMNWVRQAPGQRLEWMGLITPYNGASSYSQKFQGRVTITRDTSASTAYMELSSLRSEDTAVYYCARGGYDGRGFDYWGQGTLVTVSS

SEQ ID NO: 20 - protein, 106 aa, synthetic construct; identical to SEQ ID NO: 16

SEQ ID NO: 21 - protein, 119 aa, synthetic construct; identical to SEQ ID NO: 3

SEQ ID NO: 22 - protein, 106 aa, synthetic construct
DIQMTQSPSTLSASVGDRVTITCSASSSVSYMHWYQQKPGKAPKLLIYDTSKLASGVPSRFSGSGSGTEFTLTISSLQPDDFATYYCQQWSKHPLTFGQGTKLEIK

SEQ ID NO: 23 - protein, 122 aa, synthetic construct
QVQLQESGGGSVQAGGSLKLSCAASGRGFSRYAMGWFRQAPGQDREFVATISWTNSTDYADSVKGRFAISRDNAKNTAYLQMNSLKPEDTAVYYCAADKWASSTRSIDYDYWGQGIQVTVSS

SEQ ID NO: 24 - protein, 122 aa, synthetic construct
QVQLVDSGGGVVQPGRSLKLSCAASGRGFSRYAMGWFRQAPGKGREYVATISWTNSTDYADSVKGRFTISRDNAKNTAYLQMNSLRAEDTAVYYCAADKWASSTRSIDYDYWGKGTTVTVSS

SEQ ID NO: 25 - protein, 122 aa, synthetic construct
QVQLVDSGGGVVQPGRSLRLSCAASGRGFSRYAMGWFRQAPGKDREYVATISWTNSTDYADSVKGRFTISRDNSKNTAYLQMNSLRAEDTAVYYCAADKWASSTRSIDYDYWGQGTTVTVSS

SEQ ID NO: 26 - protein, 122 aa, synthetic construct
QVQLVDSGGGVVQPGRSLRLSCAASGRGFSRYAMGWFRQAPGKGLEYVATISWTNSTDYADSVKGRFTISRDNSKNTLYLQMNSLRAEDTAVYYCAADKWASSTRSIDYDYWGKGTTVTVSS

SEQ ID NO: 27 - protein, 122 aa, synthetic construct
QVQLVDSGGGVVQPGRSLRLSCAASGRGFSRYAMGWFRQAPGKGLEYVATISWTNSTDYADSVKGRFTISRDNSKNTLYLQMNSLRAEDTAVYYCAADKWASSTRSIDYDYWGQGTTVTVSS

SEQ ID NO: 28 - protein, 122 aa, synthetic construct
QVQLVDSGGGVVQPGRSLRLSCAASGRGFSRYAMGWFRQAPGKGLEYVATISWTNSTDYADSVKGRFTISRDNSKNTLYLQMNSLRAEDTAVYYCAADKWASSTRSIDYDYWGQGTMVTVSS

SEQ ID NO: 29 - protein, 122 aa, synthetic construct
QVQLVDSGGGVVQPGRSLKLSCAASGRGFSRYAMGWFRQAPGKDREFVATISWTNSTDYADSVKGRFTISRDNAKNTAYLQMNSLRAEDTAVYYCAADKWASSTRSIDYDYWGKGTTVTVSS

SEQ ID NO: 30 - protein, 122 aa, synthetic construct
QVQLVDSGGGVVQPGRSLKLSCAASGRGFSRYAMGWFRQAPGQDREFVATISWTNSTDYADSVKGRFTISRDNAKNTAYLQMNSLRAEDTAVYYCAADKWASSTRSIDYDYWGKGTTVTVSS

SEQ ID NO: 31 - protein, 122 aa, synthetic construct
QVQLVDSGGGVVQPGRSLKLSCAASGRGFSRYAMGWFRQAPGKDREFVATISWTNSTDYADSVKGRFTISRDNSKNTAYLQMNSLRAEDTAVYYCAADKWASSTRSIDYDYWGQGTMVTVSS

SEQ ID NO: 32 - protein, 122 aa, synthetic construct
QVQLVDSGGGVVQPGRSLKLSCAASGRGFSRYAMGWFRQAPGKDREFVATISWTNSTDYADSVKGRFTISRDNAKNTAYLQMNSLRAEDTAVYYCAADKWASSTRSIDYDYWGQGTMVTVSS

SEQ ID NO: 33 - protein, 122 aa, synthetic construct
QVQLVDSGGGVVQPGRSLKLSCAASGRGFSRYAMGWFRQAPGKDREFVATISWTNSTDYADSVKGRFTISRDNAKNTAYLQMNSLRAEDTAVYYCAADKWASSTRSIDYDYWGQGTTVTVSS

SEQ ID NO: 34 - protein, 122 aa, synthetic construct
QVQLVDSGGGVVQPGRSLKLSCAASGRGFSRYAMGWFRQAPGKDREFVATISWTNSTDYADSVKGRFTISRDNSKNTAYLQMNSLRAEDTAVYYCAADKWASSTRSIDYDYWGQGTTVTVSS

SEQ ID NO: 35 - protein, 122 aa, synthetic construct
QVQLVDSGGGVVQPGRSLKLSCAASGRGFSRYAMGWFRQAPGQGREYVATISWTNSTDYADSVKGRFTISRDNAKNTAYLQMNSLRAEDTAVYYCAADKWASSTRSIDYDYWGKGTTVTVSS

SEQ ID NO: 36 - protein, 122 aa, synthetic construct
QVQLVDSGGGVVQPGRSLRLSCAASGRGFSRYAMGWFRQAPGKDREFVATISWTNSTDYADSVKGRFTISRDNAKNTAYLQMNSLRAEDTAVYYCAADKWASSTRSIDYDYWGKGTTVTVSS

SEQ ID NO: 37 - protein, 122 aa, synthetic construct
QVQLVDSGGGVVQPGRSLRLSCAASGRGFSRYAMGWFRQAPGKDREFVATISWTNSTDYADSVKGRFTISRDNSKNTAYLQMNSLRAEDTAVYYCAADKWASSTRSIDYDYWGKGTTVTVSS

SEQ ID NO: 38 - protein, 122 aa, synthetic construct
QVQLVDSGGGVVQPGRSLRLSCAASGRGFSRYAMGWFRQAPGKDREFVATISWTNSTDYADSVKGRFTISRDNSKNTAYLQMNSLRAEDTAVYYCAADKWASSTRSIDYDYWGQGTMVTVSS

SEQ ID NO: 39 - protein, 122 aa, synthetic construct
QVQLVDSGGGVVQPGRSLRLSCAASGRGFSRYAMGWFRQAPGKDREFVATISWTNSTDYADSVKGRFTISRDNSKNTAYLQMNSLRAEDTAVYYCAADKWASSTRSIDYDYWGQGTTVTVSS

SEQ ID NO: 40 - protein, 122 aa, synthetic construct
QVQLVDSGGGVVQPGRSLRLSCAASGRGFSRYAMGWFRQAPGKDREFVATISWTNSTDYADSVKGRFTISRDNSKNTLYLQMNSLRAEDTAVYYCAADKWASSTRSIDYDYWGQGTMVTVSS

SEQ ID NO: 41 - protein, 122 aa, synthetic construct
QVQLVDSGGGVVQPGRSLKLSCAASGRGFSRYAMGWFRQAPGKDREYVATISWTNSTDYADSVKGRFTISRDNSKNTAYLQMNSLRAEDTAVYYCAADKWASSTRSIDYDYWGQGTMVTVSS

SEQ ID NO: 42 - protein, 122 aa, synthetic construct
QVQLVDSGGGVVQPGRSLKLSCAASGRGFSRYAMGWFRQAPGKDREYVATISWTNSTDYADSVKGRFTISRDNAKNTAYLQMNSLRAEDTAVYYCAADKWASSTRSIDYDYWGQGTMVTVSS

SEQ ID NO: 43 - protein, 122 aa, synthetic construct
QVQLVDSGGGVVQPGRSLKLSCAASGRGFSRYAMGWFRQAPGKDREYVATISWTNSTDYADSVKGRFTISRDNAKNTAYLQMNSLRAEDTAVYYCAADKWASSTRSIDYDYWGQGTTVTVSS

SEQ ID NO: 44 - protein, 122 aa, synthetic construct
QVQLVDSGGGVVQPGRSLKLSCAASGRGFSRYAMGWFRQAPGKDREYVATISWTNSTDYADSVKGRFTISRDNSKNTAYLQMNSLRAEDTAVYYCAADKWASSTRSIDYDYWGQGTTVTVSS

SEQ ID NO: 45 - protein, 122 aa, synthetic construct
QVQLVDSGGGVVQPGRSLRLSCAASGRGFSRYAMGWFRQAPGKGREYVATISWTNSTDYADSVKGRFTISRDNAKNTAYLQMNSLRAEDTAVYYCAADKWASSTRSIDYDYWGKGTTVTVSS

SEQ ID NO: 46 - protein, 122 aa, synthetic construct
QVQLVDSGGGVVQPGRSLRLSCAASGRGFSRYAMGWFRQAPGKGREYVATISWTNSTDYADSVKGRFTISRDNSKNTAYLQMNSLRAEDTAVYYCAADKWASSTRSIDYDYWGKGTTVTVSS

SEQ ID NO: 47 - protein, 122 aa, synthetic construct
QVQLVDSGGGVVQPGRSLRLSCAASGRGFSRYAMGWFRQAPGKDREYVATISWTNSTDYADSVKGRFTISRDNSKNTAYLQMNSLRAEDTAVYYCAADKWASSTRSIDYDYWGQGTMVTVSS